(19) United States
(12) Patent Application Publication
(10) Pub. No.: US 2022/0349815 A1
(43) Pub. Date: Nov. 3, 2022

(54) APPARATUS AND METHODS TO PRODUCE SOIL MAPS

(71) Applicants: Penelope Nagel, San Diego, CA (US); Brian Zamudio, La Jolla, CA (US); Chris Todter, Point Loma, CA (US)

(72) Inventors: Penelope Nagel, San Diego, CA (US); Brian Zamudio, La Jolla, CA (US); Chris Todter, Point Loma, CA (US)

(21) Appl. No.: 17/735,023

(22) Filed: May 2, 2022

Related U.S. Application Data
(60) Provisional application No. 63/201,483, filed on Apr. 30, 2021.

(51) Int. Cl. G01N 21/31; G01N 1/08; G01N 1/28; G01N 1/38; G01N 33/24; B65G 17/20

(52) U.S. Cl. CPC G01N 21/31; G01N 1/08; G01N 1/286; G01N 1/38; G01N 33/24; B65G 17/20; B65G 2201/0238

(57) ABSTRACT

Disclosed are improved soil mapping methods and apparatus that enable a user to efficiently analyze soil and create accurate soil maps to enable the user to determine soil content. The apparatus includes a probe cylinder, an extractor cylinder, a collector assembly, a first stepper motor, a roller assembly, a mixing motor, a contact probe, a cleaning brush motor, a bracket assembly for a mix motor, brush motor, and probe, an extruded aluminum bracing, a gear track, a second stepper motor, a scraper assembly, a third stepper motor, a probe, and one or more hyperspectral sensors. The apparatus relies on less expensive, faster, and more accurate data points that provide actionable real-time information by automating the current labor-intensive soil testing process. The apparatus serves the agricultural industry by advancing precision soil mapping technology to support proactive and efficient fertilizer use. Further, the apparatus helps farmers to increase crop yields, optimize input costs, and protect the environment by supporting fertilizer efficiency.

### PRIORITY APPLICATIONS

The present invention claims priority to Provisional Patent Application 63/201,483 entitled “IMPROVED SOIL MAPPING METHODS, PROCESSES AND APPARATUS” filed Apr. 30, 2021 and is hereby incorporated by reference in its entirety as if fully set forth.

### BACKGROUND OF THE INVENTION

### Technical Field

The present invention is generally related to an improved process, methods, and machinery for analyzing the soil. More particularly, the present disclosure relates to an apparatus to accurately produce soil maps to efficiently enable the user to determine soil content.

### Background

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also be inventions.

Currently, at least in the agricultural business, inefficient fertilizer uses results in about 5.7 billion dollars of lost crop yields and 1.7 billion dollars in excess costs. Currently, prescription maps use data from grid soil sampling, yield maps, and/or satellite images of growing crops. The current methods of conventional grid soil sampling and laboratory analysis are unable to provide the level of granular information needed, are time-consuming, and are costly. Prescription maps created with grid soil sampling are labor-intensive, expensive, and imprecise. Although many modern farmers apply variable rates of fertilizer by programming their machinery with computer-generated “prescription maps” the production of the maps is costly and inefficient resulting in the slow adoption of prescription mapping technology on more farms. 1.5 billion dollars was spent on grid sampling in the US in a year and 50 hours were lost per year on a 10,000-acre farm because of the labor-intensive nature of current sampling methods. The reason why some modern farmers are still willing to try current prescription mapping methods is that under optimized prescription mapping it is estimated that farmers could expect at least 15% reduced fertilizer costs and 13% increased crop yields which equal about 7.4 billion dollars in revenues to be gained under more optimal prescription techniques.

Unfortunately, the existing methods and apparatuses of creating soil maps are inefficient, expensive, and time-consuming resulting in many farms opting out of using soil maps and just applying either the incorrect amount of feed resulting in suboptimal field performance and waste of materials.

This specification recognizes that there is a need for efficient, cost-effective, and improved methods and apparatuses for analyzing the soil. Further, there is a need for apparatuses and methods for creating more rapid and accurate soil maps to efficiently enable the user to determine soil content thus, solving the above-described problems.

Thus, in view of the above, there is a long-felt need in the agriculture industry to address the aforementioned deficiencies and inadequacies.

Further limitations and disadvantages of conventional and traditional approaches will become apparent to one having skill in the art through comparison of described systems with some aspects of the present disclosure, as set forth in the remainder of the present application and with reference to the drawings.

### SUMMARY OF THE INVENTION

An apparatus to accurately produce soil maps to efficiently enable the user to determine soil content is provided substantially, as shown in and/or described in connection with at least one of the figures, as set forth more completely in the claims.

An aspect of the present disclosure relates to an apparatus to accurately produce soil maps to efficiently enable the user to determine soil content. The apparatus includes a probe cylinder, an extractor cylinder, a collector assembly, a first stepper motor, a roller assembly, a mixing motor, a contact probe, a cleaning brush motor, a bracket assembly for a mix motor, brush motor, and probe, an extruded aluminum bracing, a gear track, a second stepper motor, a scraper assembly, a third stepper motor, a probe, and one or more hyperspectral sensors. The probe cylinder is configured to extend the probe downward into the soil to an appropriate depth, and the probe cylinder collects the sample core and then retracts to the original starting position. The first stepper motor moves the collector assembly to a position that centers the collector assembly directly under the probe. The extractor cylinder extends pushing the sample core into the collector assembly. As the extractor cylinder begins to retract the first stepper motor activates moving the collector assembly to a position directly centered under the mixing motor. The mixing motor turns on and then the second stepper motor raises the collector assembly into the mixer which pulverizes the sample core. Once the collector assembly reaches a top position, the second stepper motor reverses lowering the collector assembly to its original beginning position under the mixing motor. Then the mixing motor turns off. Further, the first stepper motor moves the collector assembly to a position centered directly under the contact probe. The second stepper motor then raises the collector assembly to a position so that the contact probe is inside the collector assembly just above the mixed sample. At this time the contact probe scans the soil sample. Once the scan is completed the second stepper motor then reverses lowering the collector assembly to its beginning position directly under the contact probe.

The first stepper motor then moves the collector assembly to a position directly center under the brush motor and over a bag holder conveyor, wherein the third stepper motor activates and opens a slide on the bottom of the collector assembly allowing the soil sample to be deposited in a bag that is situated on the bag holder conveyor directly under the collector assembly.

The brush motor turns on at the same time the second stepper motor activates raising the collector assembly upward into a brush assembly. The brush assembly consists of a fiber brush the same size as the collector assembly which cleans any soil residue off of the collector assembly. Once the collector assembly reaches its most upward position it reverses and lowers to its original starting position under the brush motor. The brush motor then stops and the third stepper motor activates shutting the bottom slide on the collector assembly. The bag holder conveyor then advances to the next bag holder ready for a new sample and repeats the process. The hyperspectral sensors are configured to detect soil samples.

In an aspect, the hyperspectral sensors are placed in controlled halogen lighting conditions.

In an aspect, the soil samples are passed under hyperspectral sensors. The result, after calibration of the hyperspectral sensors for both white reflectance and dark (no reflectance) values, and spatial averaging over the sample area was a spectral signature for each of the 48 samples, and at both the “as sent” and the “air-dried” water levels.

In an aspect, the hyperspectral sensors have a range of 400-2400 nm. The controlled halogen lighting conditions are 700 Watts and approximating daylight levels per square meter.

In an aspect, the apparatus further comprises an image collector; a database, a processor, and an output device, wherein the database is associated with confirmed spectral images; and the processor is configured to process the spectral images for adjusting the image based on field parameters, and the output device presents or displays soil map information.

Accordingly, one advantage of the present invention is that it serves the agricultural industry by advancing precision soil mapping technology to support proactive and efficient fertilizer use.

Accordingly, one advantage of the present invention is that it supports fertilizer efficiency that helps farmers increase crop yields, optimize input costs, and protect the environment.

Accordingly, one advantage of the present invention is that it provides faster, easier, more accurate methods for analyzing soil and producing soil maps.

Accordingly, one advantage of the present invention is that it provides increased data points, and also provides actionable real-time information for gathering and processing field data.

Accordingly, one advantage of the present invention is that it provides a competitive advantage over existing or non-using users and automates existing, and labor-intensive processes.

Accordingly, one advantage of the present invention is that it includes the use of spectral imaging to minimize or even ameliorate the need for labor-intensive soil chemical testing for agricultural maps.

Accordingly, one advantage of the present invention is that it determines soil content involving the use of improved sensors, improved data collection techniques, and improved processing of collected data to more precisely and more efficiently determine soil content.

Accordingly, one advantage of the present invention is that it includes the ability to be hands-free in relation to the soil and imaging can occur via the use of workers in the fields, riders on ATVs, or Tractors, flight of unmanned vehicles such as drones, or even satellite imaging. Sensors may be general spectral imaging sensors or filtered for certain minerals, lighting, or soil parameters (such as moisture or pH).

Accordingly, one advantage of the present invention is that it includes the ability to adjust the findings of the spectral images taken based on lighting, moisture, pH, type of soil, or seasonal variations.

Accordingly, one advantage of the present invention is that it includes the ability to build a predictive database library for each point confirmed through chemical testing such that a true soil fingerprint can be compared to a fingerprint database to more precisely determine the soil content conditions.

Accordingly, one advantage of the present invention is that it includes the ever-learning database that expands with each confirmed sample and enables a process for improved precision over time which may ameliorate the need for confirmatory chemical analysis testing.

Additional embodied methods relate to the speed of analysis wherein an image can be sent to the cloud or within local network storage and processed immediately in real-time such that a delivery prescription for the map grid can be coordinated and applied at the same time. An embodied example could include a sensor on the front of a tractor that images the field conditions as it slowly drives over a field, the images are collected and immediately sent to the database and processed, a prescription for the current field site is returned, and sent to a spreader on the back end of the tractor and the proper amount of materials is spread on to the field.

These features and advantages of the present disclosure may be appreciated by reviewing the following description of the present disclosure, along with the accompanying figures wherein like reference numerals refer to like parts.

### BRIEF DESCRIPTION OF DRAWINGS

The accompanying drawings illustrate the embodiments of systems, methods, and other aspects of the disclosure. Any person with ordinary skills in the art will appreciate that the illustrated element boundaries (e.g., boxes, groups of boxes, or other shapes) in the figures represent an example of the boundaries. In some examples, one element may be designed as multiple elements, or multiple elements may be designed as one element. In some examples, an element shown as an internal component of one element may be implemented as an external component in another and vice versa. Furthermore, the elements may not be drawn to scale.

Various embodiments will hereinafter be described in accordance with the appended drawings, which are provided to illustrate, not limit, the scope, wherein similar designations denote similar elements, and in which:

FIG. 1 illustrates a perspective view of an apparatus to produce a soil map, in accordance with at least one embodiment.

FIG. 2 illustrates a perspective view of a bottom portion of the apparatus, in accordance with at least one embodiment.

FIG. 3 illustrates a perspective view of a side portion of the apparatus, in accordance with at least one embodiment.

FIG. 4 illustrates a graphical representation of the measured data for the 48 samples, as determined by a contract chemical analysis lab, in accordance with at least one embodiment.

FIGS. 5A-5B illustrate the samples 1-40 in the dry state, and the photos clearly show that even though samples are from the same field at the same time they can be markedly different, in accordance with at least one embodiment.

FIG. 6 illustrates a graphical representation of the correlation between the spectra for all dry samples and wavelength (nm), in accordance with at least one embodiment.

FIG. 7 illustrates a graphical representation of the correlation between the different colored sample 12 and its spectrum, in accordance with at least one embodiment.

FIG. 8 illustrates a graphical representation of a spectral plot, sample 35 is the bold red trace and sample 36 is the bold green trace, and there is a substantial difference in overall reflectance due to the soil texture, in accordance with at least one embodiment.

FIG. 9 illustrates a graphical representation of a plot of sample 1 both wet and dry, in accordance with at least one embodiment.

FIG. 10 illustrates a graphical representation of the same reflectance degradation and shift with carefully assessed moisture levels, in accordance with at least one embodiment.

FIGS. 11-15 illustrate graphical representations of correlation or fit values for each of the 48 samples plotted against their measured values, in accordance with at least one embodiment.

FIG. 16 illustrates a graphical representation of a comparison of chemical and spectral for potassium, in accordance with at least one embodiment.

FIG. 17 illustrates a graphical representation of a comparison of chemical and spectral for phosphorus, in accordance with at least one embodiment.

FIG. 18 illustrates a graphical representation of a comparison of chemical and spectral for nitrogen, in accordance with at least one embodiment.

FIG. 19 illustrates a graphical representation of a comparison of chemical and spectral for Ph, in accordance with at least one embodiment.

FIG. 20 illustrates a graphical representation of a comparison of chemical and spectral for organic matter, in accordance with at least one embodiment.

FIG. 21 illustrates an area plot of average nitrogen based on chemical analysis, in accordance with at least one embodiment.

FIG. 22 illustrates an area plot of average nitrogen based on spectral data, in accordance with at least one embodiment.

FIG. 23 illustrates an area plot of phosphorus-based on chemical analysis, in accordance with at least one embodiment.

FIG. 24 illustrates an area plot of phosphorus-based on spectral analysis, in accordance with at least one embodiment.

FIG. 25 illustrates an area plot of potassium based on chemical analysis, in accordance with at least one embodiment.

FIG. 26 illustrates an area plot of potassium based on spectral analysis, in accordance with at least one embodiment.

FIG. 27 illustrates an area plot of average phosphorus based on chemical analysis, in accordance with at least one embodiment.

FIG. 28 illustrates an area plot of average Ph based on spectral analysis, in accordance with at least one embodiment.

FIG. 29 illustrates an area plot of organic material from chemical analysis, in accordance with at least one embodiment.

FIG. 30 illustrates an area plot of organic material from spectral analysis, in accordance with at least one embodiment.

FIG. 31 illustrates a perspective view of a soil texture triangle, in accordance with at least one embodiment.

### DETAILED DESCRIPTION

The present disclosure is best understood with reference to the detailed figures and description set forth herein. Various embodiments have been discussed with reference to the figures. However, those skilled in the art will readily appreciate that the detailed descriptions provided herein with respect to the figures are merely for explanatory purposes, as the methods and systems may extend beyond the described embodiments. For instance, the teachings presented and the needs of a particular application may yield multiple alternative and suitable approaches to implement the functionality of any detail described herein. Therefore, any approach may extend beyond certain implementation choices in the following embodiments.

References to “one embodiment,” “at least one embodiment,” “an embodiment,” “one example,” “an example,” “for example,” and so on indicate that the embodiment(s) or example(s) may include a particular feature, structure, characteristic, property, element, or limitation but that not every embodiment or example necessarily includes that particular feature, structure, characteristic, property, element, or limitation. Further, repeated use of the phrase “in an embodiment” does not necessarily refer to the same embodiment.

Methods of the present invention may be implemented by performing or completing manually, automatically, or a combination thereof, selected steps or tasks. The term “method” refers to manners, means, techniques, and procedures for accomplishing a given task including, but not limited to, those manners, means, techniques, and procedures either known to, or readily developed from known manners, means, techniques and procedures by practitioners of the art to which the invention belongs. The descriptions, examples, methods, and materials presented in the claims and the specification are not to be construed as limiting but rather as illustrative only. Those skilled in the art will envision many other possible variations within the scope of the technology described herein.

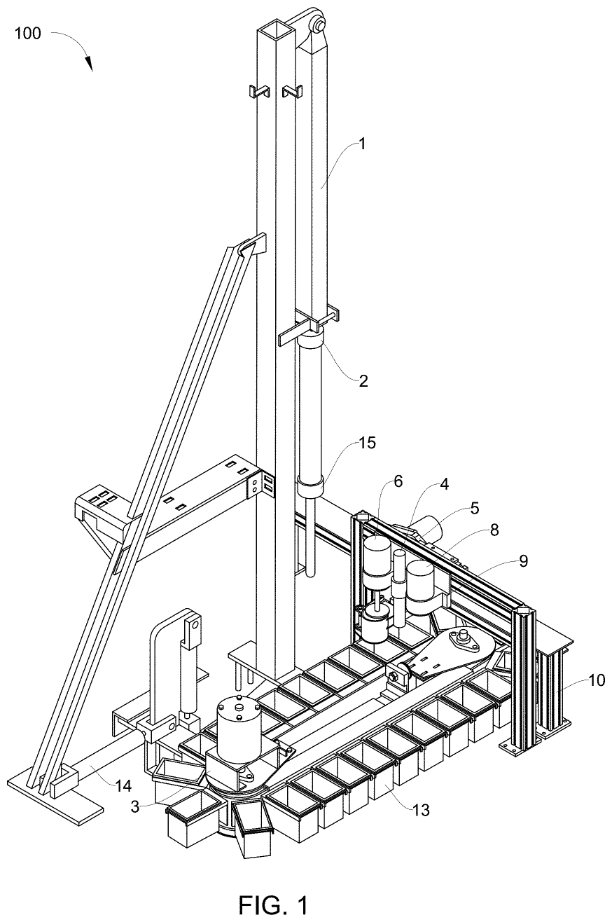

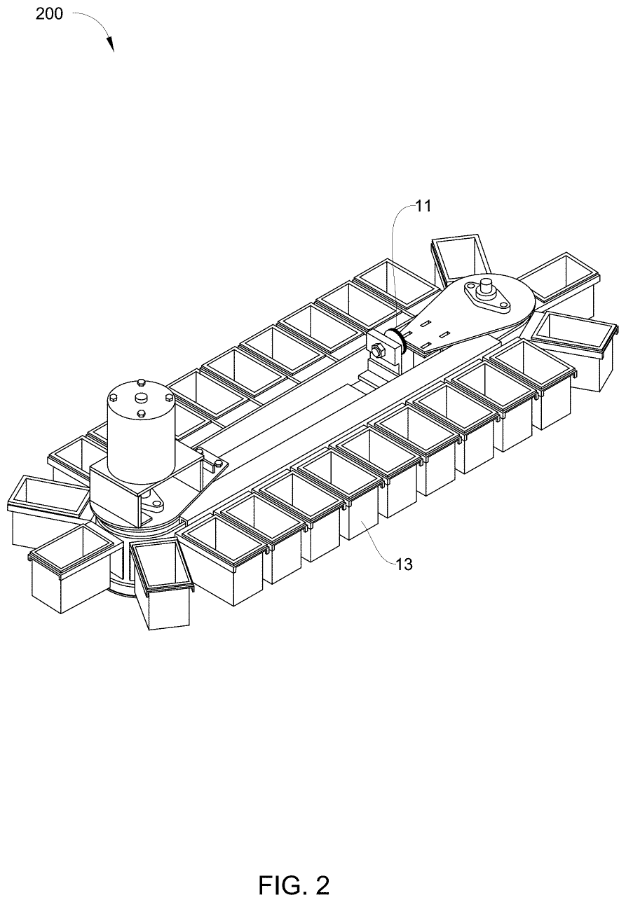

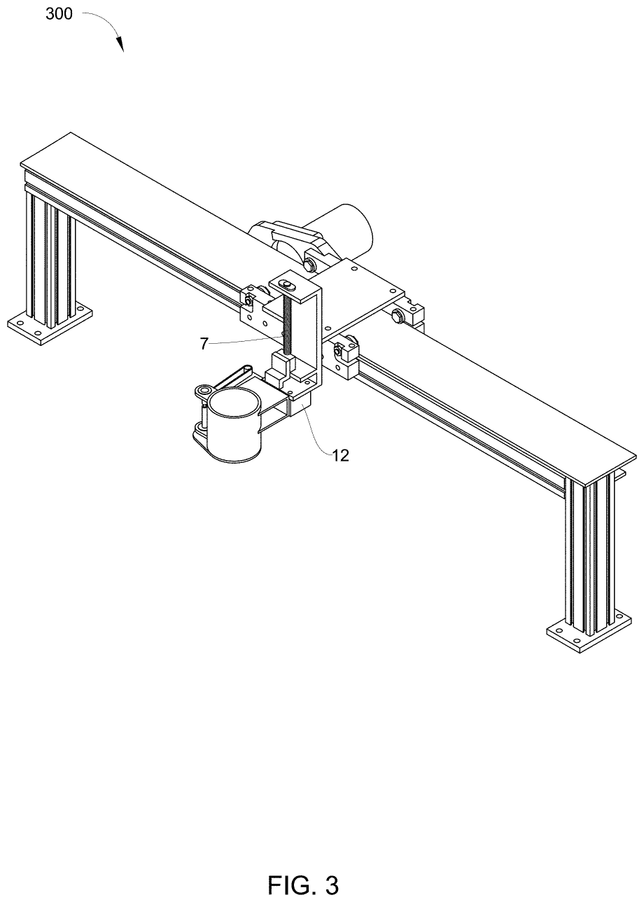

FIG. 1 illustrates a perspective view of an apparatus (100) to accurately produce soil maps to efficiently enable the user to determine soil content, in accordance with at least one embodiment. The apparatus (100) includes a probe cylinder (1), an extractor cylinder (2), a collector assembly (3), a first stepper motor (4), a roller assembly (5), a mixing motor (6), a contact probe (7) (shown in FIG. 3), a cleaning brush motor (8), a bracket assembly for mix motor, brush motor, and probe (9), an extruded aluminum bracing (10), a gear track (11) (shown in FIG. 2), a second stepper motor (12) (shown in FIG. 3), a scraper assembly (13) (shown in FIG. 2), a third stepper motor (14), a probe (15), one or more hyperspectral sensors, an image collector, a database, and a processor. FIG. 1 is explained in conjunction with FIGS. 2-3. The probe cylinder (1) is configured to extend the probe (15) downward into the soil to an appropriate depth, and the probe cylinder (1) collects the sample core and then retracts to the original starting position. The first stepper motor (4) moves the collector assembly (3) to a position that centers the collector assembly (3) directly under the probe (15). The extractor cylinder (2) extends pushing the sample core into the collector assembly (3). As the extractor cylinder (2) begins to retract the first stepper motor (4) activates moving the collector assembly (3) to a position directly centered under the mixing motor (6).

FIG. 2 illustrates a perspective view of a bottom portion (200) of the apparatus, in accordance with at least one embodiment. FIG. 3 illustrates a perspective view of a side portion (300) of the apparatus, in accordance with at least one embodiment. The mixing motor (6) turns on and then the second stepper motor (12) raises the collector assembly (3) into the mixer which pulverizes the sample core. Once the collector assembly (3) reaches a top position, the second stepper motor (12) reverses lowering the collector assembly (3) to its original beginning position under the mixing motor. Then the mixing motor (6) turns off. Further, the first stepper motor (4) moves the collector assembly (3) to a position centered directly under the contact probe (7). The second stepper motor (12) then raises the collector assembly (3) to a position so that the contact probe (7) is inside the collector assembly (3) just above the mixed sample. At this time the contact probe (7) scans the soil sample. Once the scan is completed the second stepper motor (12) then reverses lowering the collector assembly (3) to its beginning position directly under the contact probe (7).

The first stepper motor (4) then moves the collector assembly (3) to a position directly center under the brush motor (8) and over a bag holder conveyor, wherein the third stepper motor (14) activates and opens a slide on the bottom of the collector assembly (3) allowing the soil sample to be deposited in a bag that is situated on the bag holder conveyor directly under the collector assembly (3). The brush motor (6) turns on at the same time the second stepper motor (12) activates raising the collector assembly (3) upward into a brush assembly. The brush assembly consists of a fiber brush the same size as the collector assembly (3) which cleans any soil residue off of the collector assembly (3). Once the collector assembly (3) reaches its most upward position it reverses and lowers to its original starting position under the brush motor (8). The brush motor (8) then stops and the third stepper motor (14) activates shutting the bottom slide on the collector assembly (3). The bag holder conveyor then advances to the next bag holder ready for a new sample and repeats the process.

The hyperspectral sensors are configured to detect soil samples. In an embodiment, the hyperspectral sensors are placed in controlled halogen lighting conditions. In an embodiment, the soil samples are passed under hyperspectral sensors. The result, after calibration of the hyperspectral sensors for both white reflectance and dark (no reflectance) values, and spatial averaging over the sample area was a spectral signature for each of the 48 samples, and at both the “as sent” and the “air-dried” water levels. In an embodiment, the hyperspectral sensors have a range of 400-2400 nm. The controlled halogen lighting conditions are 700 Watts and approximating daylight levels per square meter. In an embodiment, the apparatus (100) further includes an image collector; a database, a processor, and an output device, wherein the database is associated with confirmed spectral images; and the processor is configured to process the spectral images for adjusting the image based on field parameters, and the output device presents or displays soil map information.

Generally, the use of spectral analysis for identifying soil components requires a greater understanding of the previous shortcomings of previous uses have been. The greatest challenge for utilizing spectral analysis as in the present invention is the ability to try and control for the various variances seen outside of a laboratory environment. In comparison, the chemical analysis techniques are typically performed under controlled conditions. However, the shortcomings of chemical analysis regarding expense and time have been addressed in the background section of this specification. The present embodiments create processes and methods for using spectral imaging in a way that can account for the variances associated with ambient conditions and mimic the analytical precision of chemical analysis.

There have been several tests done on soil using spectral analysis. They range from lab condition (consistent controlled lighting, and controlled moisture content) analysis to aerial data acquisition from 1800 ft and satellite analysis. The objectives have been to determine the effects of moisture content, effects of soil type, effects of soil texture, and grain size, to attempt to quantify total carbon content, and nitrogen levels, to be able to classify soils by type, etc.

More testing has been done in a laboratory setting to minimize lighting and moisture effects, with reasonable results. Some testing has been done in the field with poorer results due to environmental and soil conditions.

Analysis methods have ranged from simple regression to more complicated mathematical modeling.

Additionally, the following is a list of macro and micronutrients that the present invention covers with the initial targets of: Nitrogen Phosphorus Organic Material (OM), Potassium, pH, Moisture, and other elements including; Mg, Ca, Oxygen content (OC), S, Zn, B, Fe, Mn, Cu, Na, etc.

Challenges associated with precision farming: There are ongoing economic pressures in production agriculture to increase crop yields. However, high grain yield production comes at a cost of applying significant quantities of various agricultural inputs, i.e., nutrients, pesticides, and irrigation. In traditional farming systems, producers attempt to apply these inputs at a uniform rate across a given field. However, due to inherent spatial variability in fields, not all areas may require the same levels of input. Although the spatial and temporal variability of yield-limiting factors discussed above has been recognized for a long-time farmers continued to manage their fields uniformly because they lacked the technology to manage for variability. With the introduction of new precision farming technologies such as global positioning systems (GPS), geographic information systems (GIS), remote sensing, and variable rate application technology (VRT) farmers now can manage their fields site specifically.

As more producers become aware of precision farming technology, they are asking how precision farming can improve their productivity and profitability. Variable-rate fertilizer application is promoted by industry as a way to increase efficiency and improve production. Environmentally, it seems correct to vary the amount of fertilizer in relation to crop need; however, this will not appeal to farmers unless economic gain from VRT can be demonstrated. When inputs are inexpensive relative to the value of the crop produced, farmers will logically apply inputs at a level to ensure all areas of the field receives an adequate level of the input. This has in some instances slowed the adoption of more efficient VRT systems. However, as these relationships change it will provide a tipping point for Precision Agriculture adoption. Recent years have seen increased price volatility for both farm inputs and products. Few inputs have experienced such dramatic price fluctuations relative to grain as nitrogen (N), phosphorus (P), and potassium (K) fertilizers. Variable-rate fertilizer application can decrease required fertilizer application while maintaining and even increasing crop yields.

Producers have limited experience with this new technology and equipment and need unbiased information to determine whether VRT is a feasible option for their farming operations. The objectives of the present embodiments are to address the challenges facing precision farming by presenting new tools to address these challenges to enable farmers to better utilize these effective solutions to high fertilizer costs and low commodity prices.

The present specification provides variable rate application with grid soil sampling and prescription maps.

Field level studies have shown that organic C, total N, NO3-N, P, and K have spatial dependence and variation. Using the ratio of the nugget to total semivariance to classify spatial dependence, organic C, total N, NO3-N, P, and K were strongly spatially dependent. Other studies have concluded that N, P, and K uptake and crop response vary spatially within fields. Welsh et al. (1999) reported significant yield increases where 30% more additional N was applied to historically higher-yielding parts of the field. Kachanoski et al. (1996) showed that optimal levels of N, P, and K fertilization have spatial variability. The maximum yield increase and the economic yield increase over the check yield with no N, P, and K applied were both strongly correlated with spatially optimal economic return of N (r=0.70 to 0.88). Variable-rate application technologies enable farmers to adjust fertilizer rates to reflect these variations.

Accurate prescription maps are essential for effective VRT fertilizer application Grid soil sampling has most frequently been used to develop these prescription maps. Past users have indicated several technical and economic limitations associated with this approach. For one, there is a need to keep the number of samples to a minimum while still allowing a reasonable level of map quality. However, the optimum grid density may depend on the coefficient of variation. In many cases, where the spatial distribution is rather complex, much finer grid densities than those currently used commercially are required to produce accurate prescription maps. It has been indicated that a common commercial grid sampling scale of 100 m was grossly inadequate and that sampling at greater intensities only modestly improved prediction accuracy which would not justify the increase in sampling cost. This data suggests that the use of the field average fertility values in their research field was not substantially worse than grid sampling. Schloeder et al. (2001) demonstrated that spatial interpolation of grid sampled data with a limited sample size (n=46) was mostly inappropriate. For most of their data sets the inability to predict, could be attributed to either spatially independent data, limited data, sample spacing, extreme values, or erratic behavior. Whelan et al. (1996) reported that in fields with less than 100 samples only very simple geostatistical methods such as inverse distance are appropriate. Sample sizes of 100 to 500 are needed for geostatistical methods such as kriging. Several interpolation techniques, such as ordinary kriging, lognormal kriging, and inverse distance weighting, have been studied and it has been found that the best geostatistical methods to use depended on unique spatial properties in each field and could not be predicted in advance. McBratney and Pringle, (1999) reported that grid sampling at 20 to 30 m is generally needed when applying site-specific management at a resolution of 20 by 20 m.

As can be seen, no one grid size or interpolation technique adequately describes the variability that exists in fields of a diverse population. If one fails to sample at a fine enough resolution to capture the spatial correlation in crop nutrient data, the interpolation methods and the variable rate application maps developed from those methods will not be valid or accurate. However, the cost associated with grid sampling to the intensity required for accurate maps will be prohibitive in many cases.

The present specification provides better tools for VRT to provide efficient remote sensing.

Due to the technical and economic limitations associated with grid soil sampling described above, better tools are needed to fully realize the potential VRT technologies can provide. Remote sensing is a technology that can be used to obtain various spatial layers of information about soil and crop conditions. It allows detection and/or characterization of an object, series of objects, or landscape without physical contact. Typically, remote sensing is conducted by positioning a sensor above the object (target) being observed. Platforms that support the sensors vary, depending on the altitude above the target. Today three main observation platforms are used to collect remote sensing data: UAV-based, aircraft-based, and satellite-based. Ground-based sensors also have been used for certain specific applications and research studies.

Sensors commonly used for remote sensing are part of either passive or active systems. Active systems, such as radar, supply their source of energy to illuminate target surfaces. Passive systems, like a common photo camera, detect reflected solar energy. Although several concepts involving active systems have been developed at the research level, primarily passive systems are used in commercial applications related to site-specific management.

During the last half of the century, remote sensing instrumentation developed from simple optical systems into complex digital sensors, allowing rapid and high-quality scanning of the Earth's surface. Computation algorithms have been developed to process remotely sensed data and to produce different types of images. Spatial, spectral, and temporal resolutions are the main characteristics of any remote sensing system.

Spatial resolution refers to the smallest area (pixel) that can be distinguished in the image. Each pixel becomes a data point. As with photography, the distance between the sensor and the target, as well as the viewing angle, defines the field of view (i.e., the size of the area represented by a single image or scan). Most images and data sets used in site-specific management have spatial resolutions ranging from less than 1 meter to 20 meters or more. Smaller pixel size usually is more expensive and requires more storage space and computation power.

Spectral resolution defines the ability of the system to differentiate between levels of electromagnetic radiation across different wavelengths (portions of the spectrum). The number of sensed portions of the spectrum (bands) and their width also characterize the spectral resolution of the system. Some sensors (especially photographic) produce only black and white, color, or color infrared images, while others allow recording multispectral (typically less than 10) or hyper-spectral responses (can be more than a hundred). Panchromatic images also can be used to represent total reflectance combined from visual and near-infrared bands.

Temporal resolution refers to the time between sequential data collection events using the same source. This is especially important while studying crop growth conditions. The schedule of orbiting satellites and planned aircraft missions do not always allow obtaining data in favorable weather conditions and at the desired times.

Generally, the embodiments of the present invention are related to remote soil sensing technology but ground-based or other forms of hands-free type sensing are also contemplated.

The implementation of sustainable agricultural and environmental management requires a better understanding of the soil at increasingly finer scales for precision agriculture. As discussed, conventional grid soil sampling and laboratory analyses cannot provide this information because the spatial resolution is low, they are time-consuming and expensive. Remote soil sensing can overcome these shortcomings because the techniques facilitate the collection of larger amounts of spatial data using cheaper, simpler, and less laborious techniques. Diffuse reflectance spectroscopy using visible-near-infrared (vis-NIR) and mid-infrared (mid-IR) energies can be used to estimate soil organic carbon (OC) and soil nutrient composition. These sensors measure the amount of light that is diffusely reflected from the soil after radiation containing all the pertaining frequencies illuminates it. The parameter values cannot be directly deciphered from the vis-NIR or mid-IR spectra. To be useful quantitatively, spectra have to be exactly related to a set of known reference samples through the calibration of a prediction model, and these reference samples have to be representative of the range of soils the model is intended for. Some of the inaccuracies of calibrations may arise from the lack of sufficient absorption features, particularly in the vis-NIR, and from large soil type diversity in the calibration sets. By identifying and processing information for these factors, the present embodiments achieve the maximum generalization capacity for the calibration of a particular soil property. In addition, the embodied spatial density (one meter or less) of the remotely sensed data greatly increases the accuracy of the interpolation techniques needed to generate a robust prescription map.

Embodiments of the present invention utilize remotely sensed LANDSAT multispectral resolution crop density satellite imagery. LANDSAT is a joint USGS/NASA project with the primary objective to ensure the collection of consistently calibrated earth imagery. Temporal resolution is 16 days, the platform has developed a re-sampling routine to produce a 5-meter spatial resolution. The technology initially creates a Normalized Difference Vegetative Index (NDVI) map from the imagery. The NDVI relates the reflectance in the red region and near-infrared (NIR) region to vegetative variables such as leaf area index, canopy cover, and the concentration of total chlorophyll, which have in turn been associated with crop yield. A spatial yield potential map is then developed from the NDVI data. Remotely sensed imagery of corn canopies, particularly during mid-grain filling is highly correlated with grain yield and can offer an attractive alternative to the use of a combine yield monitor. Remotely sensed yield maps are not affected by inaccuracies (problems connected with grain flow dynamics and accurate logging of geographical position) associated with combined yield monitors. Multispectral imagery can be a useful data source for mapping crop yield as well. Correlation analysis showed that yield monitor sorghum grain yield was significantly related to the image data. The imagery data explained 92% of the grain yield variability. The use of reflectance indices, NIR/RED and NIR/GRN, derived from wheat canopy reflectance at the booting stage, is highly correlated with grain yield. The derived regression equations used to estimate grain yields also have the potential to predict yields for years to come. Accurately characterizing yield potential within a field and thus spatial N demand may be necessary for site-specific N management.

Further, the present specification provides spatial N, P, and K recommendations.

From the embodied remote soil sensing data, the data is processed and developed into variable-rate Nitrogen (N), Phosphorus (P), and Potassium (K) application maps (other nutrients or compounds may also be identified). The user inputs the N, P, and K recommendation equations appropriate for their area.

Fertilizer savings from $15.00 to $40.00+ per acre can be achieved utilizing this system as well as 3 to 5 percent yield increases. The system is also environmentally effective in reducing fertilizer over-application leading to runoff and leaching into groundwater. It is an approved enhancement practice in the NRCS conservation reserve program. By combining the embodied techniques one can create fertilizer management systems that can finally begin to fully utilize VRT's vast potential.

The following examples are intended to illustrate but not limit the invention.

### Example 1

### Soil Sample Example

As described in the below example, analysis performed while developing the embodied methods and processes included working with a farm management company and selecting a 160-acre field in western Illinois, then comparing the results with a local soils lab which grid sampled the field using their standard 2.5-acre grid.

Each sample was split in half. Half of each sample went to the soil lab for conventional analysis the other half was sent for hyperspectral sensor analysis.

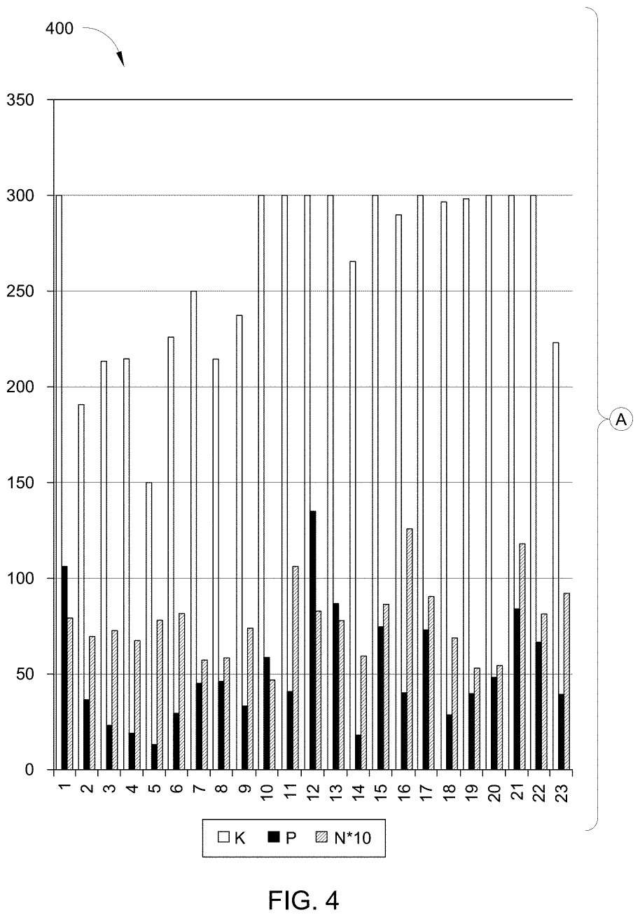

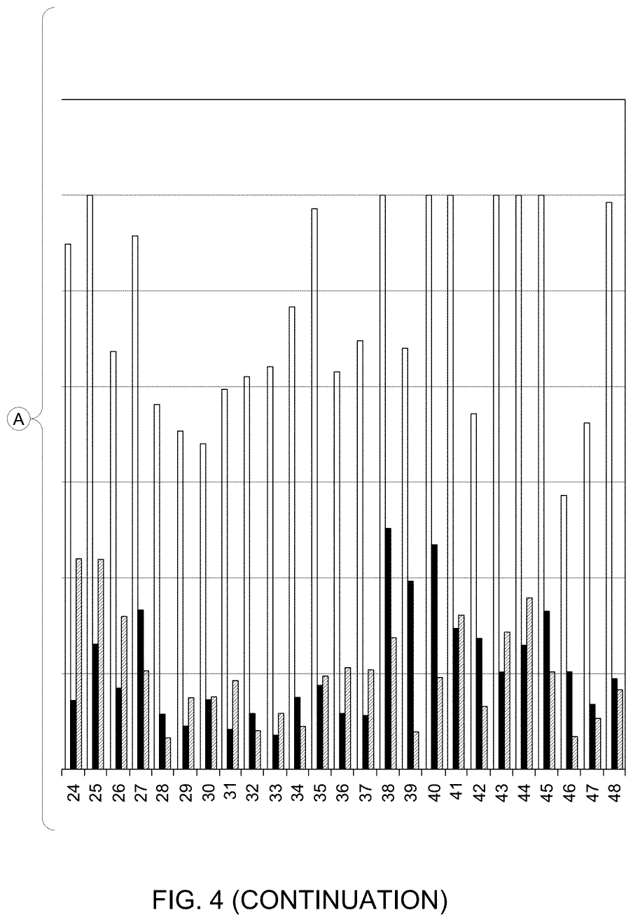

The following non-limiting example shown in FIG. 4 represents a soil sampling report and accompanying data as part of a soil sampling library. FIG. 4 illustrates a graphical representation (400) of the measured data for the 48 samples, as determined by a contract chemical analysis lab, in accordance with at least one embodiment. There is a good spread of measured values for each of the N, P, and K. The only troubling feature is the fact that the K values saturate at 300, which makes the correlation to the spectra more difficult and somewhat reliable.

The objective of the exemplary report was to assess the results of a first lab test conducted on 48 samples.

Test Methodology:

Soil samples were collected on-site in a grid spaced throughout the field at the appropriate timing and during the agricultural cycle such that representative levels of Nitrogen (N), Potassium (K), and Phosphorous (P) were present. The samples were stored in sealed paper bags, then sent to a contract agricultural laboratory to have the levels of the N, K, and P assessed via chemical testing.

The samples were also assessed in a lab environment using spectrometry techniques. The samples were separated into two equal groups and the first group was then air-dried. The moisture content of each group was not measured.

Utilizing two hyperspectral sensors (covering the range of 400-2400 nm) in controlled halogen lighting conditions (700 Watts, approximating daylight levels per square meter), the samples were passed under the sensors. The result, after calibration of the sensors for both white reflectance and dark (no reflectance) values, and spatial averaging over the sample area was a spectral signature for each of the 48 samples, and at both the “as sent” and the “air-dried” water levels.

The results shown in FIG. 4 show the measured data for the 48 samples, as determined by the contract chemical analysis lab. There is a good spread of measured values for each of the N, P, and K.

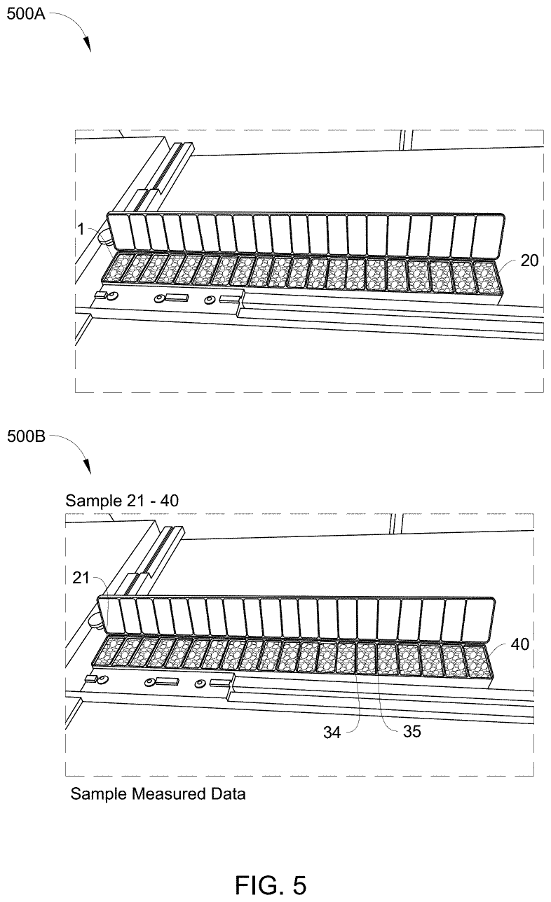

FIGS. 5A-5B illustrate perspective views (500A and 500B) of the samples 1-40 in the dry state, and the photos clearly show that even though samples are from the same field at the same time they can be markedly different, in accordance with at least one embodiment.

The SPECTIR Test results were presented in a plot of the spectra for all 48 dry samples. The notable features were:

1. A blip at 987 nm was due to a crossover between the VISNIR sensor and the SWIR sensor and had nothing to do with the actual reflectance.

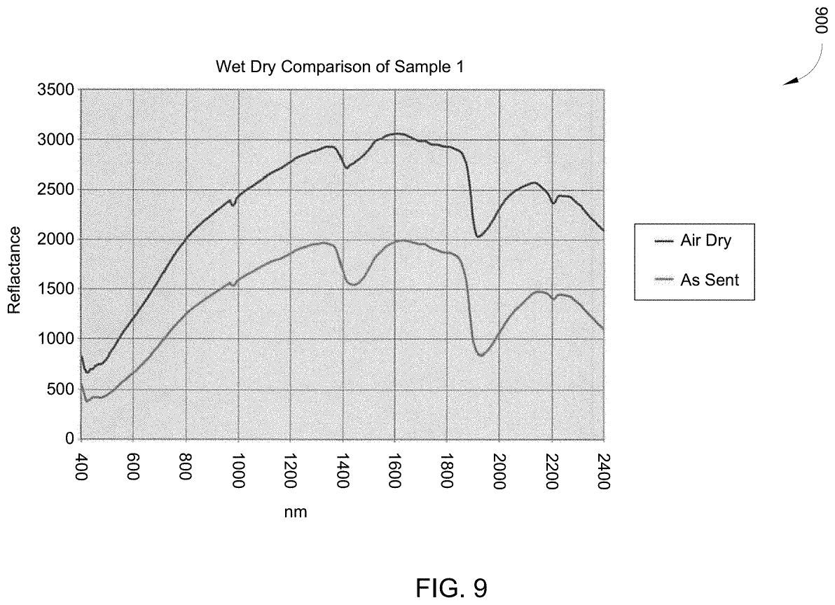

2. The large dips at about 1400 and 1900 are water absorption features. This can be seen in FIG. 9 which shows sample 1 both wet and dry.

3. While all the spectra have the same general shape there is quite a spread in values. Most of the NPK features are fairly fine grain wavelength resolution, not the larger overall level shifts. The larger overall level shifts have been determined to be caused by a combination of moisture and soil texture or grain sizes. In addition, under ambient lighting conditions (as opposed to the test lab conditions), the light level and color also contribute to this general shape shifting.

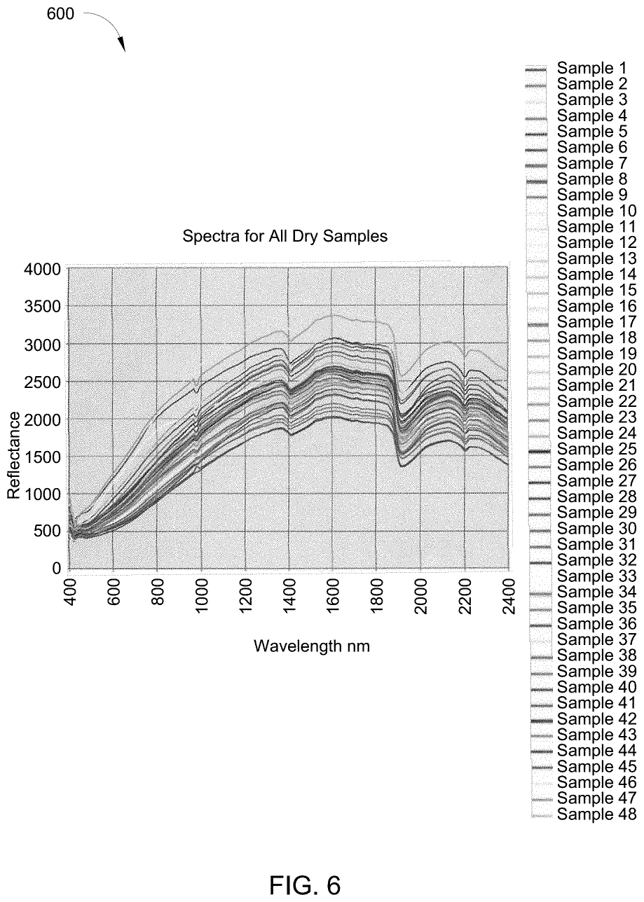

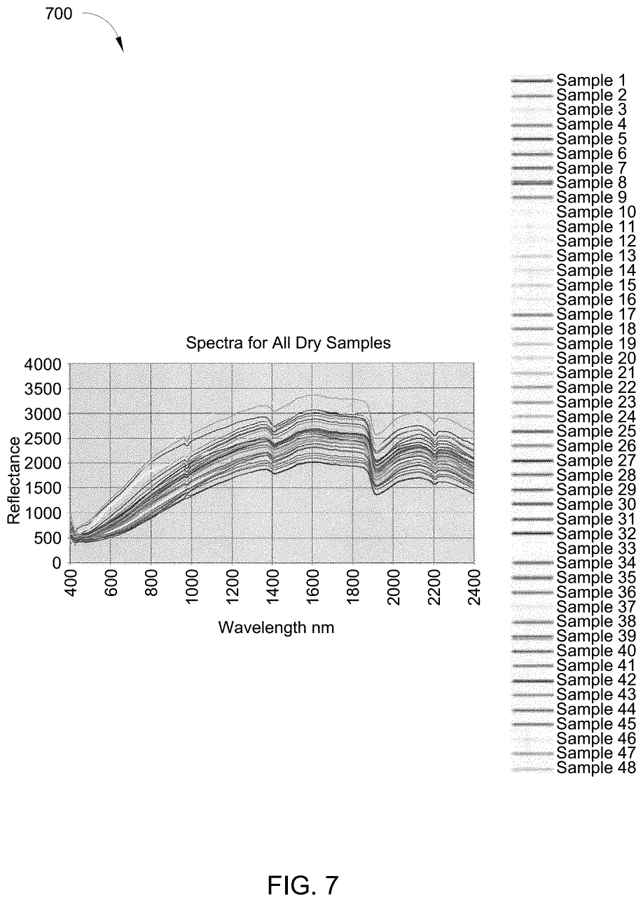

FIG. 6 illustrates a graphical representation (600) of the correlation between the spectra for all dry samples and wavelength (nm), in accordance with at least one embodiment. FIG. 7 illustrates a graphical representation (700) of the correlation between the different colored sample 12 and its spectrum as a bold yellow trace, in accordance with at least one embodiment.

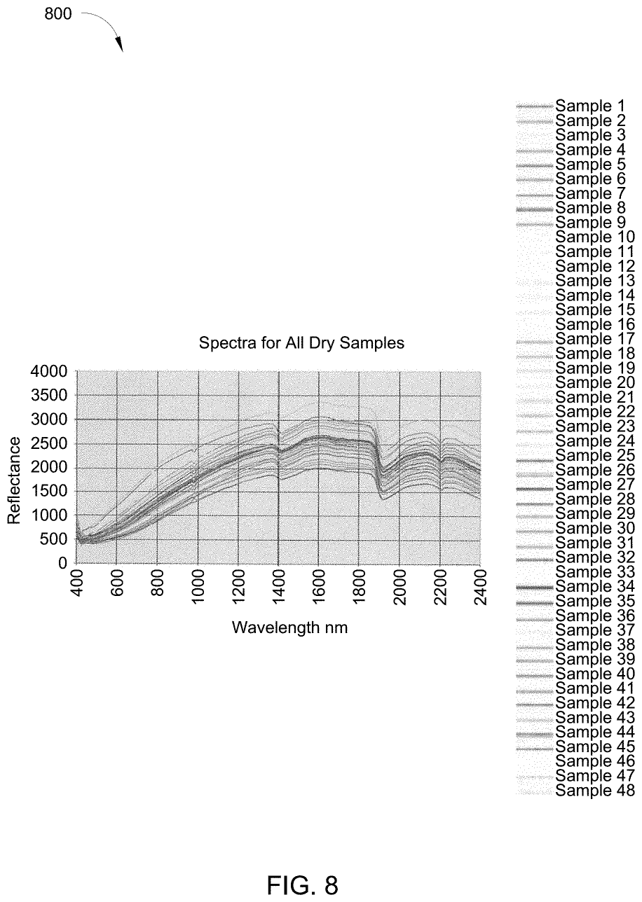

There is also an interesting and telling difference between samples 34 and 35 shown in FIG. 6, primarily in the texture or grain size of the samples. The lab measurements of these samples show relatively similar amounts of the N, P, and K measurements compared with the overall spread. FIG. 8 illustrates a graphical representation (800) of a spectral plot, sample 35 is the bold red trace and sample 36 is the bold green trace, and there is a substantial difference in overall reflectance due to the soil texture, in accordance with at least one embodiment.

The effect of moisture level is highlighted in FIG. 9. FIG. 9 illustrates a graphical representation (900) of a plot of sample 1 both wet and dry, in accordance with at least one embodiment. There is a large drop in signal level with the addition of some level of moisture. The water absorption features at 1400 and 1900 are much stronger with added moisture. As mentioned above, the actual moisture levels are unknown.

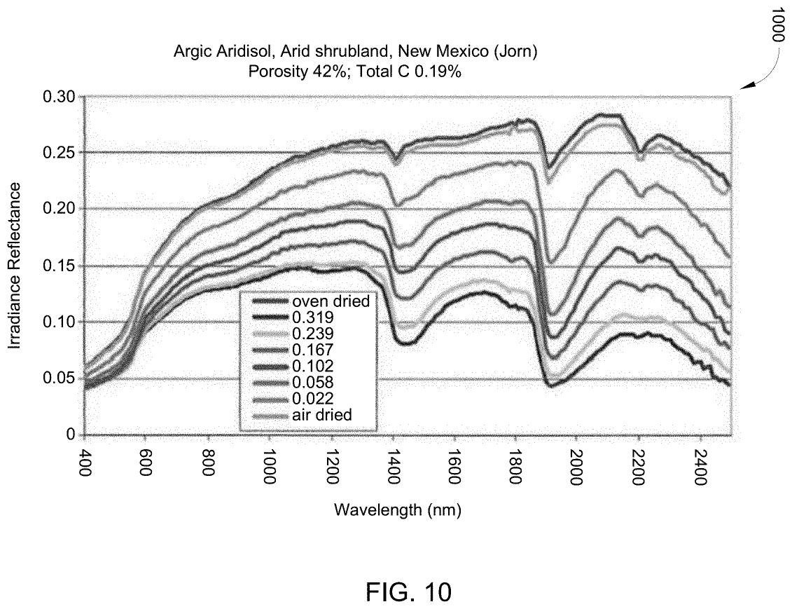

FIG. 10 illustrates a graphical representation (1000) from a paper by Lobell and Asner, 2002 showing the same reflectance degradation and shift with carefully assessed moisture levels, in accordance with at least one embodiment. This data shows the potential for modeling the moisture-induced curve shifting using the water reflectance and absorption features as a basis for normalizing response.

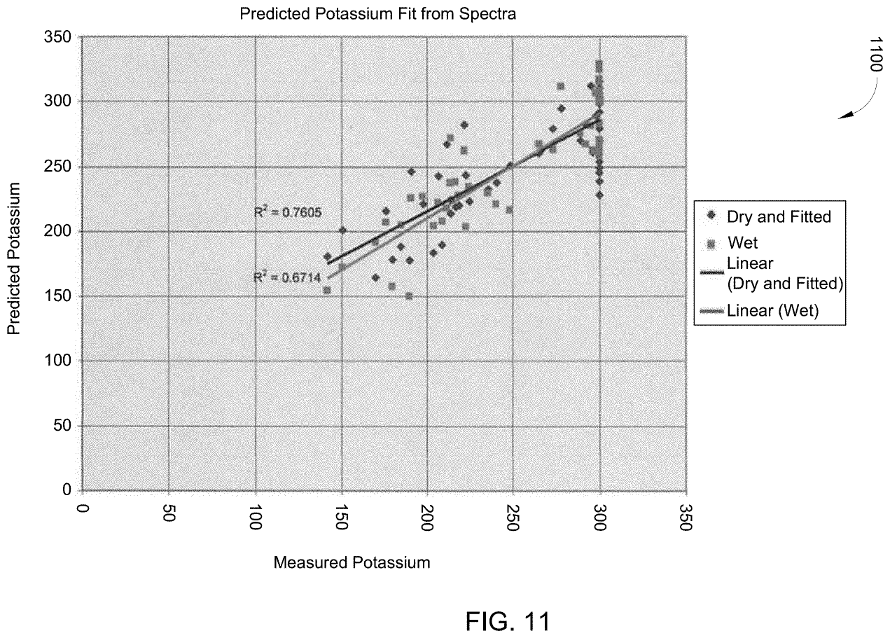

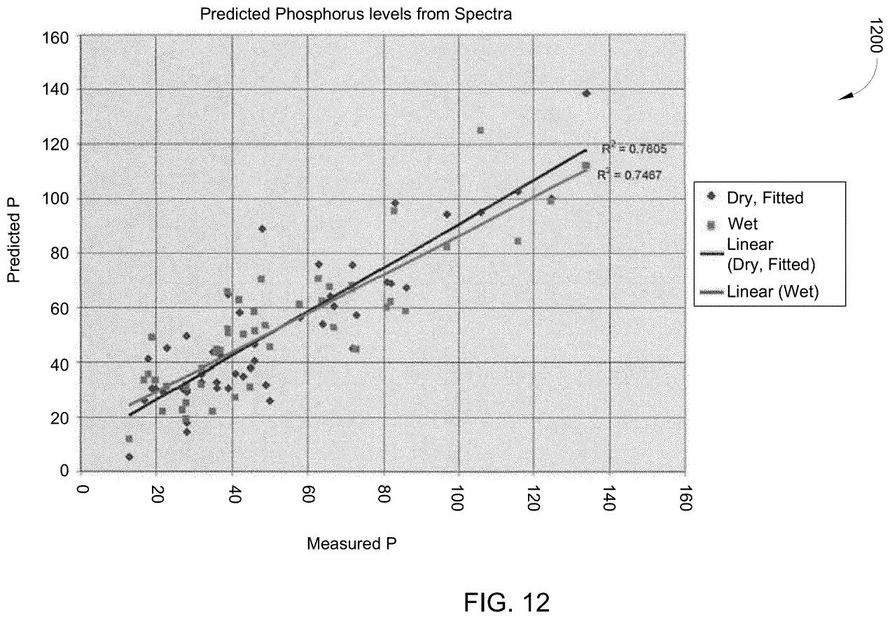

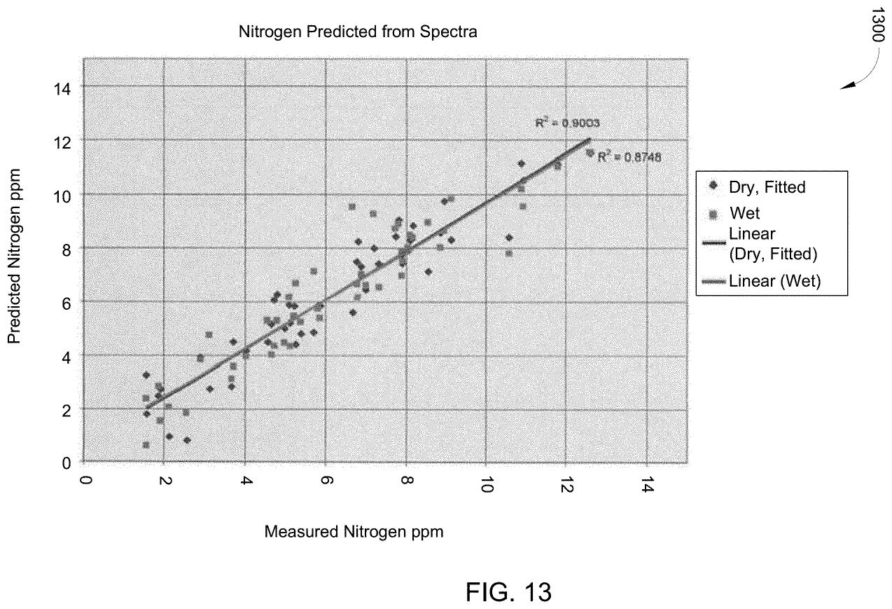

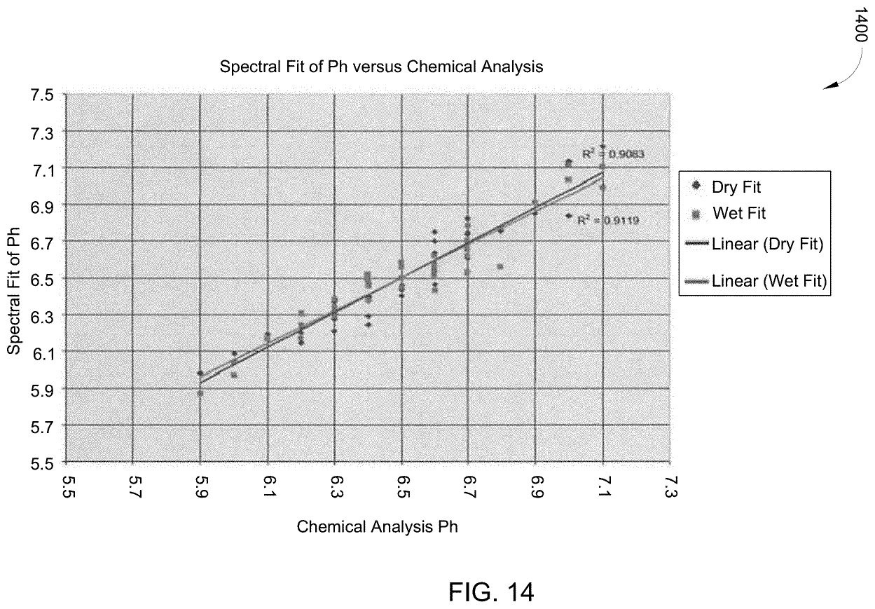

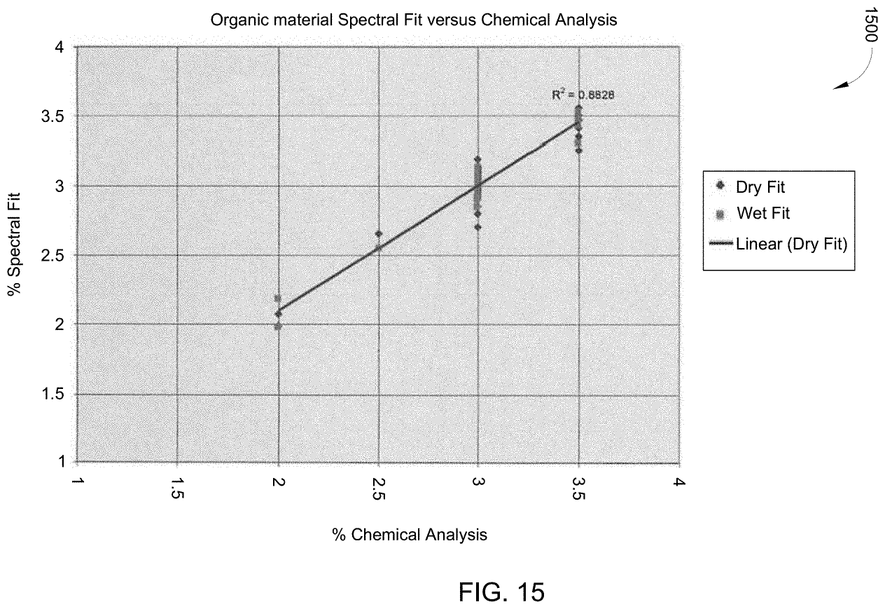

The basic approach is to do a single regression fit for each element. By doing a simple non-linear iterative regression, a correlation value is determined for each of the N, P, K elements, and the Ph and organic material (OM) levels. This works very well for fitting the dry data, or for fitting the wet data, but it becomes immediately obvious that you can not predict wet values using dry coefficients or vice versa. FIGS. 11-15 illustrate graphical representations (1100, 1200, 1300, 1400, and 1500) of correlation or fit values for each of the 48 samples plotted against their measured values, in accordance with at least one embodiment. The blue points are fitting the dry data and the pink are from fitting the wet data.

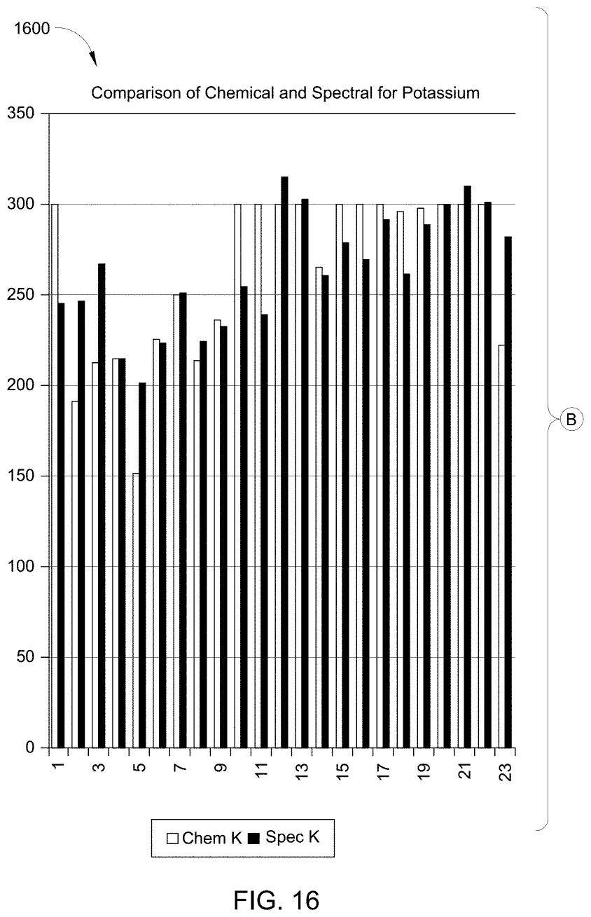

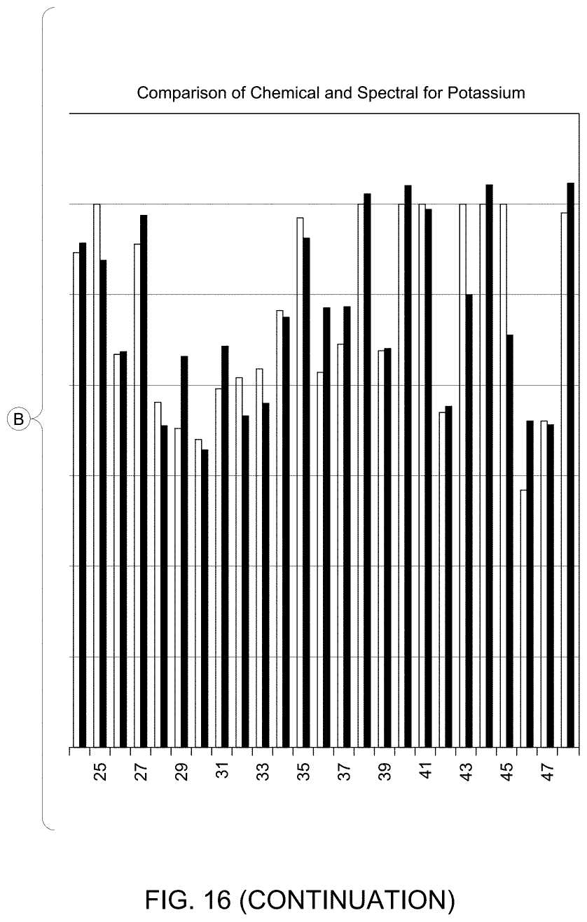

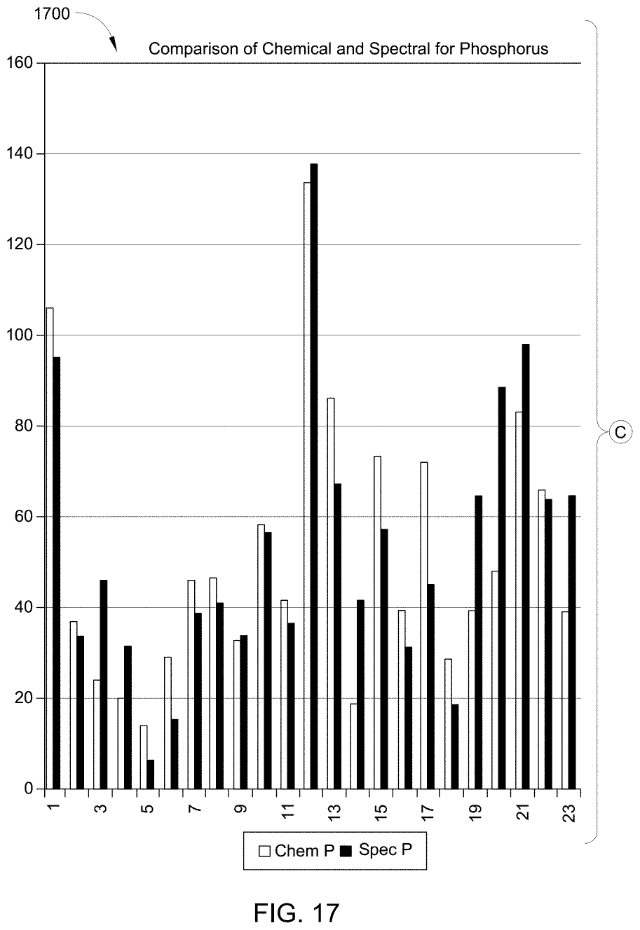

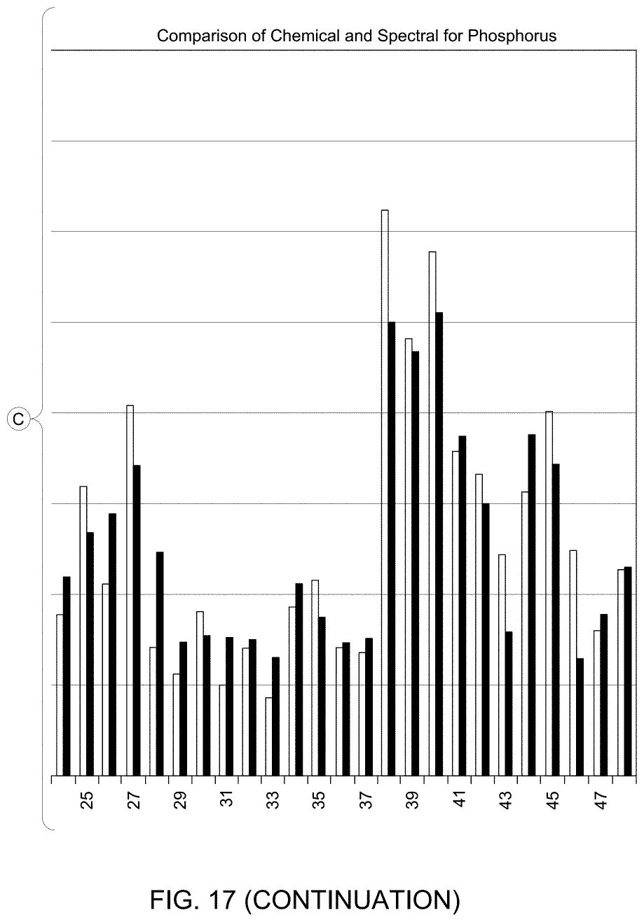

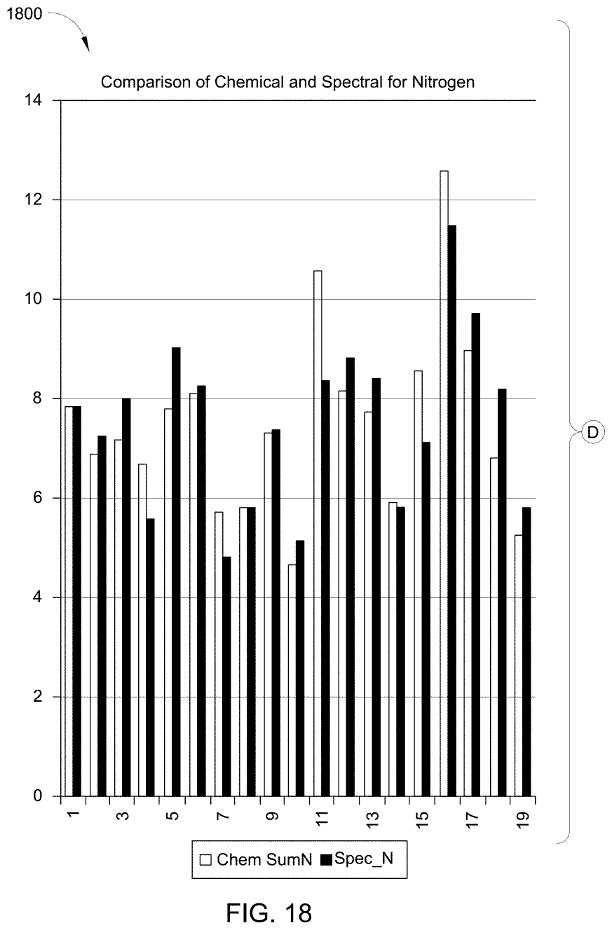

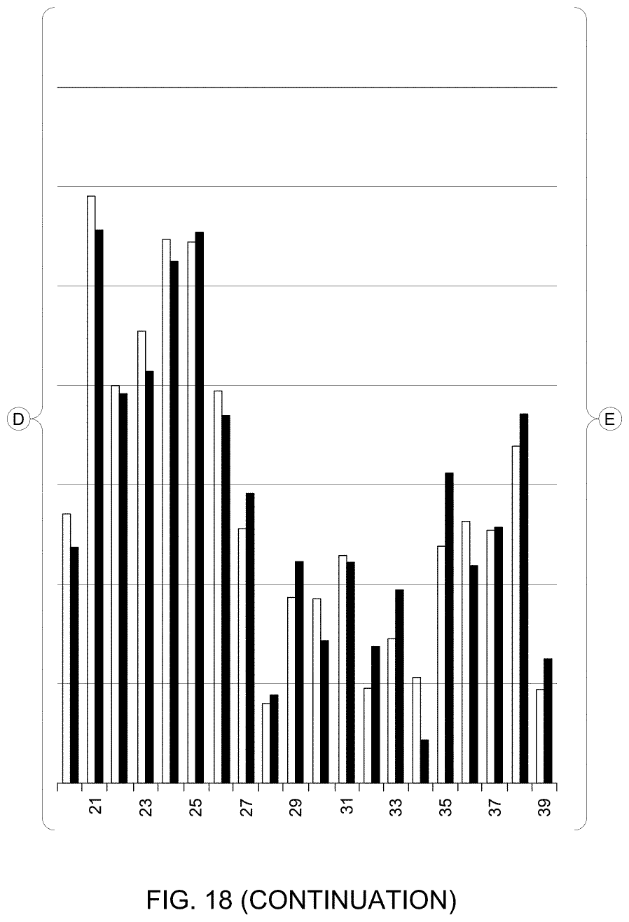

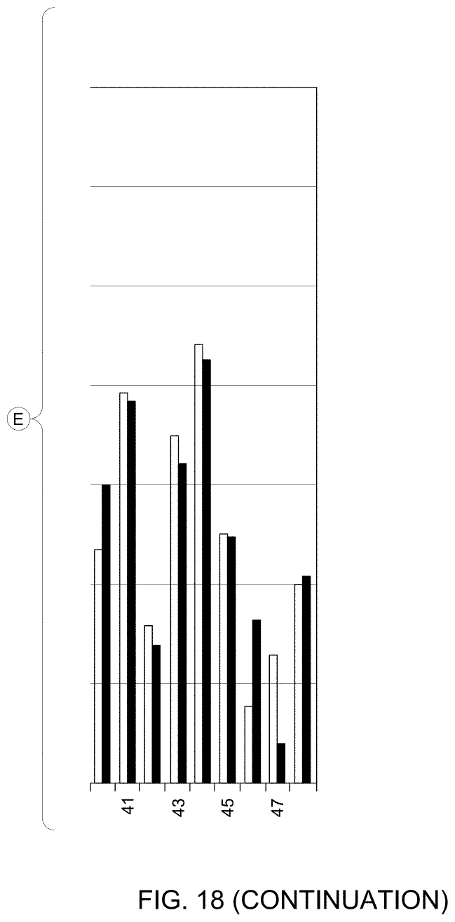

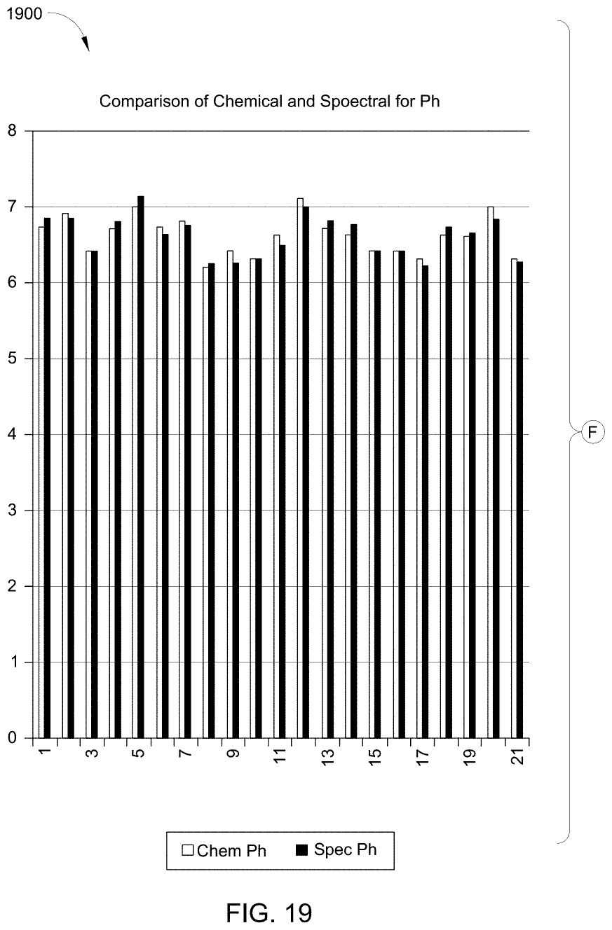

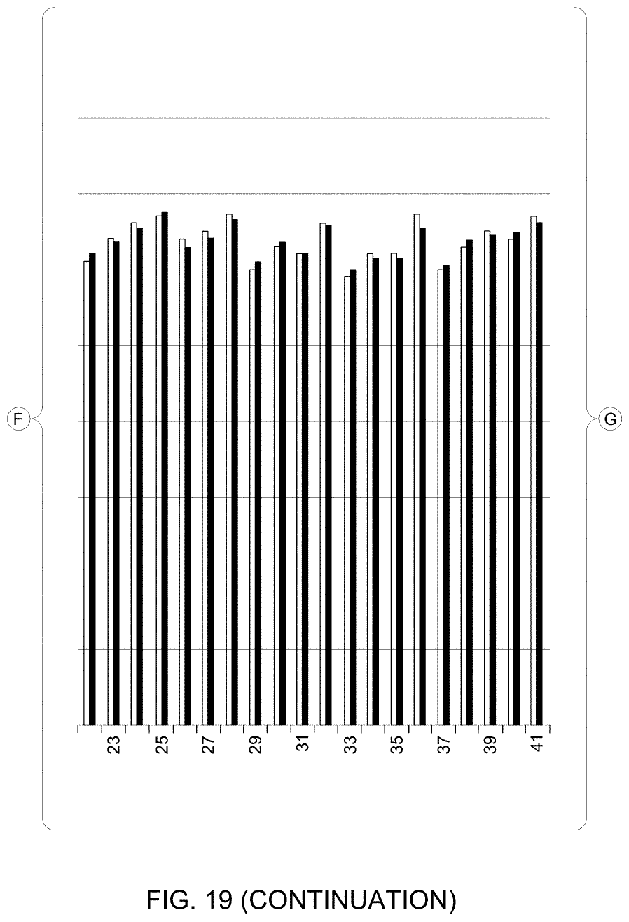

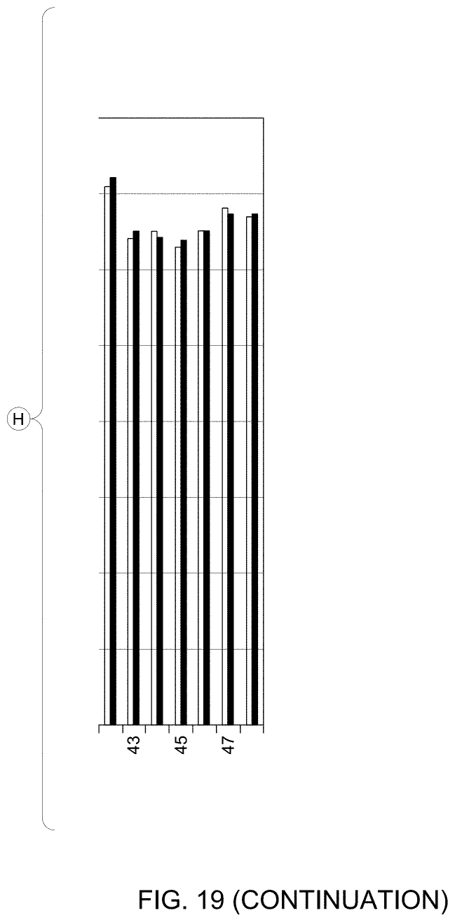

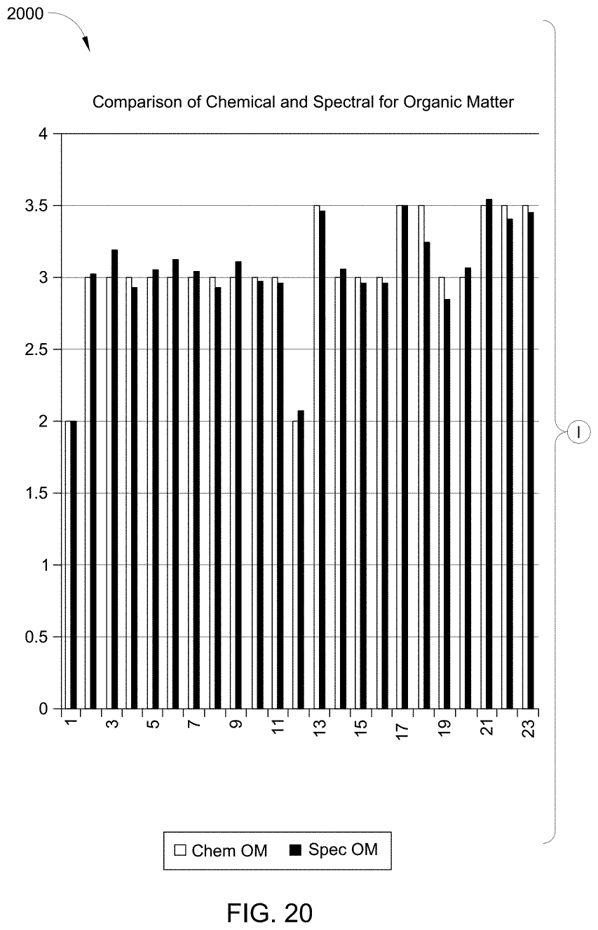

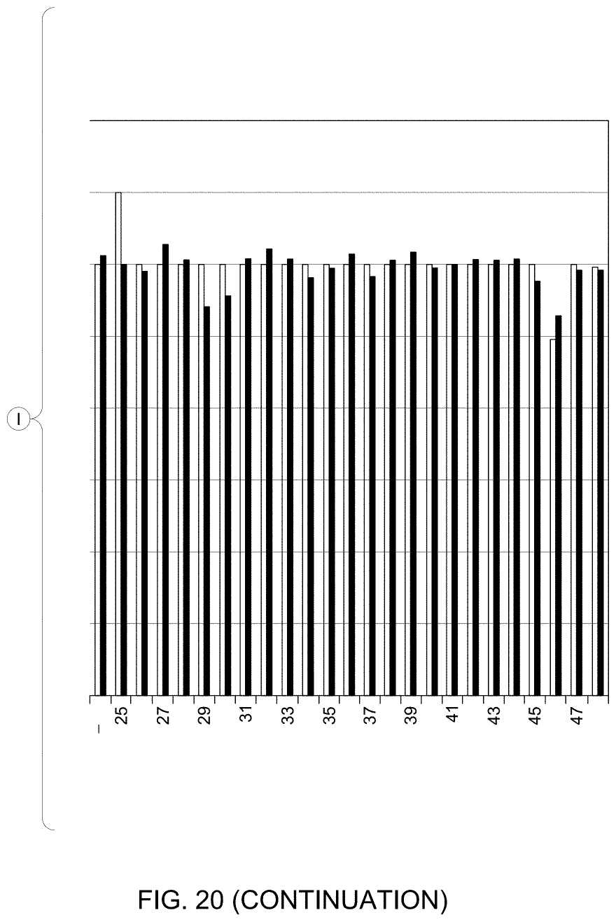

For all of these fits, N, P, K, Ph, and OM, wet, dry the correlation coefficients range between 0.82, and 0.89, which means that these coefficients do a good job of predicting the measured levels, provided we have fir with the measured levels in the beginning. In other words, if we had an additional 10 samples from the same fields which we hadn't used in the fitting procedure, but measured their reflectance as we did for the fitted samples, we could predict the actual vales very well. FIG. 16-FIG. 20 depict bar chart direct comparison plots of N, P, K, Ph, and OM in bar graph form. FIG. 16 illustrates a graphical representation (1600) of a comparison of chemical and spectral for potassium, in accordance with at least one embodiment. FIG. 17 illustrates a graphical representation (1700) of a comparison of chemical and spectral for phosphorus, in accordance with at least one embodiment. FIG. 18 illustrates a graphical representation (1800) of a comparison of chemical and spectral for nitrogen, in accordance with at least one embodiment. FIG. 19 illustrates a graphical representation (1900) of a comparison of chemical and spectral for Ph, in accordance with at least one embodiment. FIG. 20 illustrates a graphical representation (2000) of a comparison of chemical and spectral for organic matter, in accordance with at least one embodiment. Comparison plots of the spatial distribution of the three elements (N, P, and K), pH, and OM generated from the chemical analysis of the 48 soil samples are presented below. Each page has both the chemical analysis derived plot and the plot generated from the spectral data correlation for comparison. As apparent, the character and trends of the correlated spectral plots show excellent agreement with the plots coming directly from the chemical analysis.

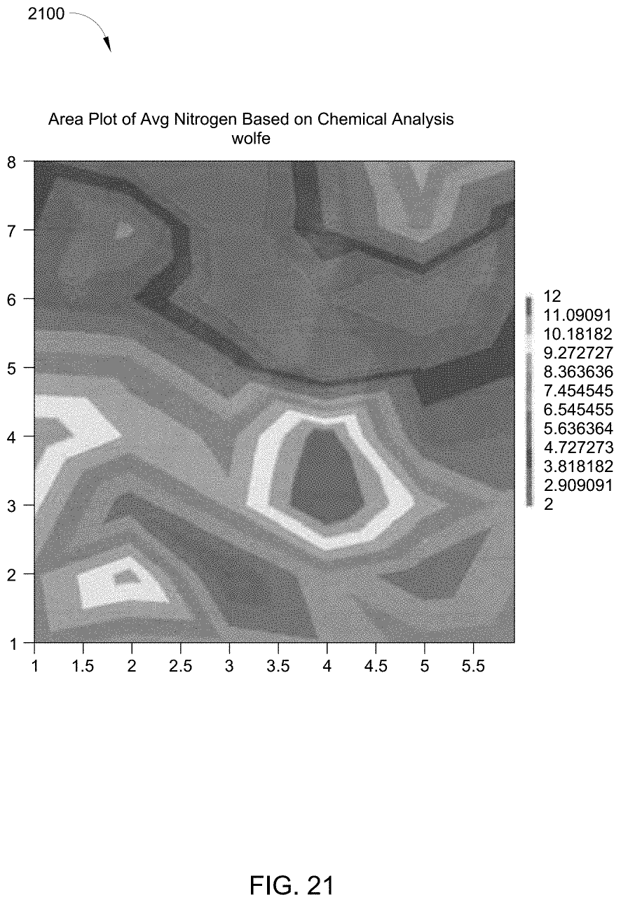

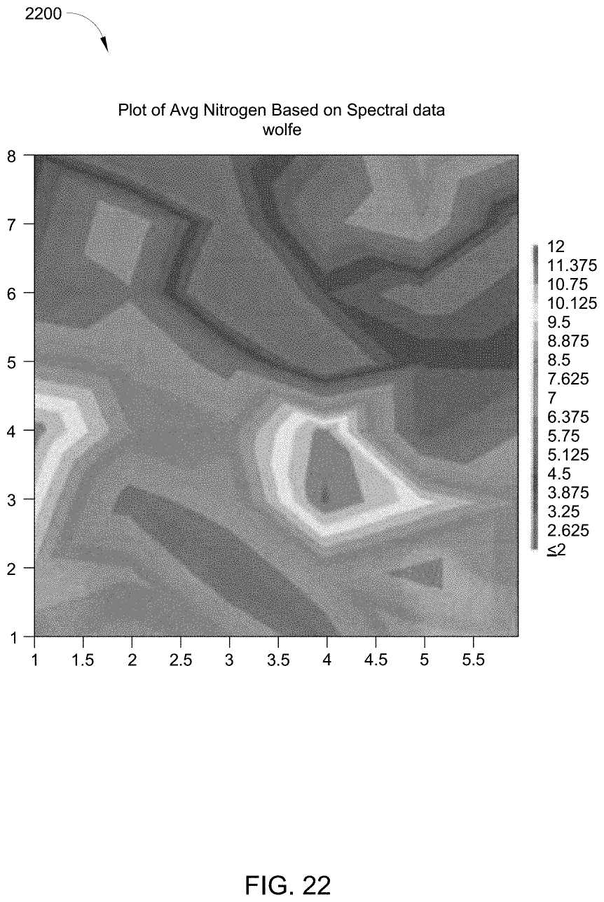

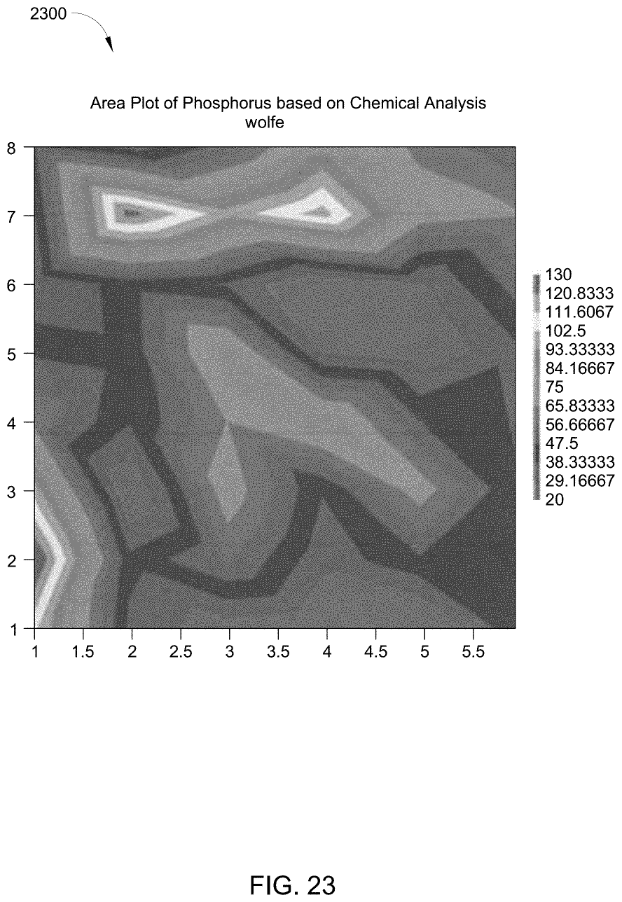

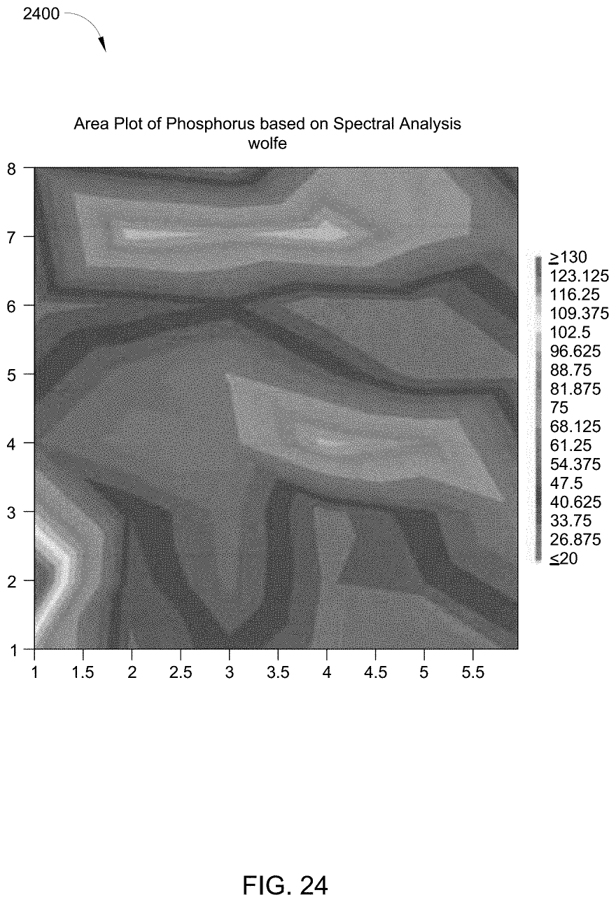

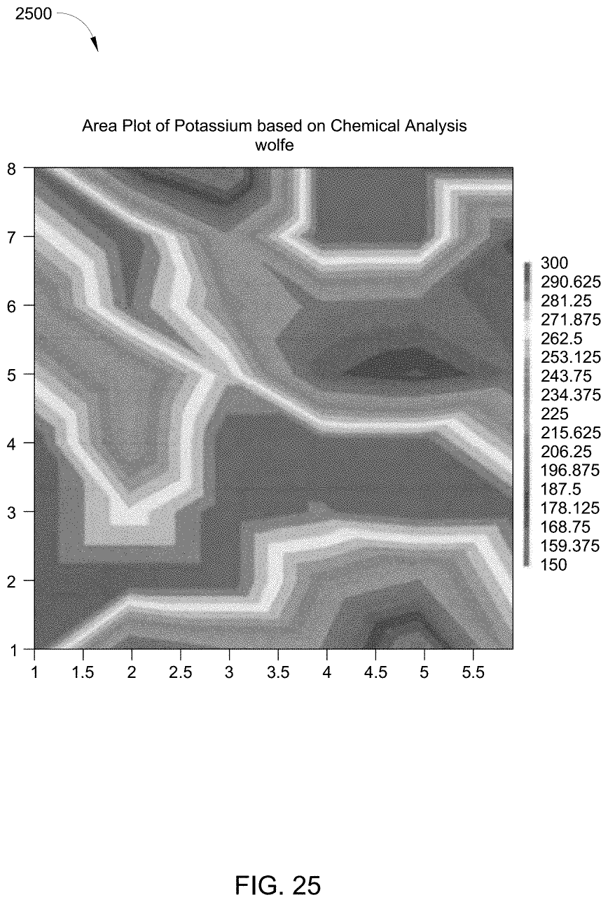

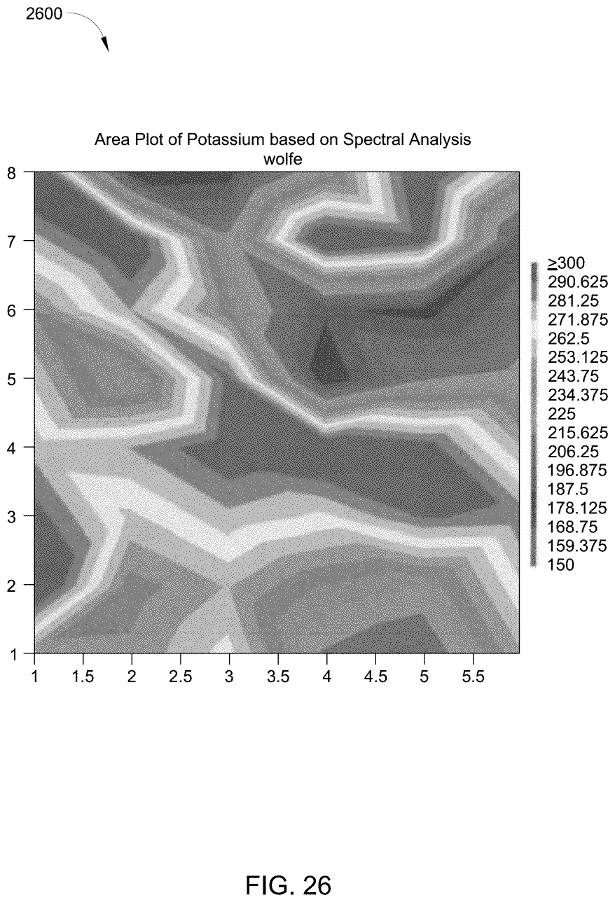

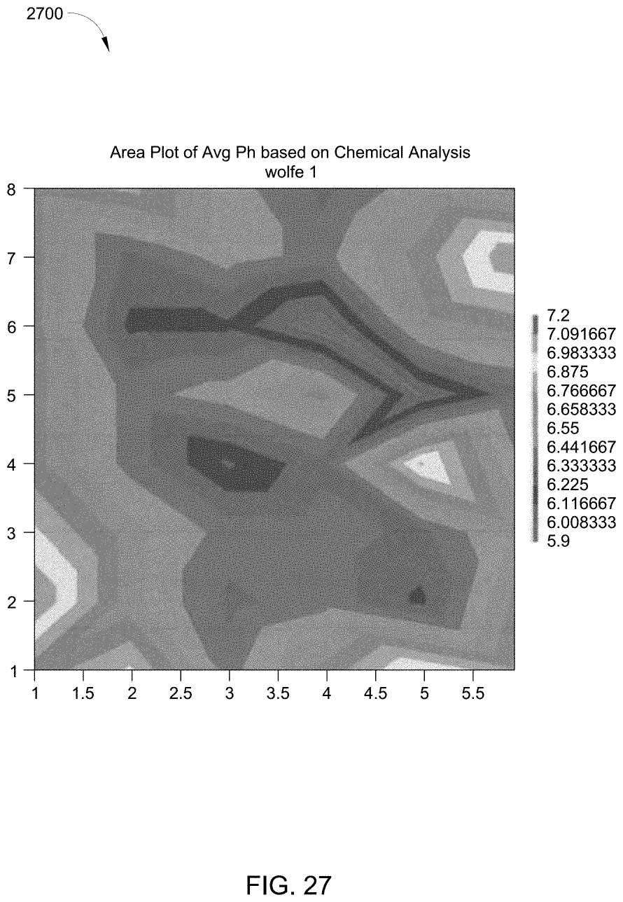

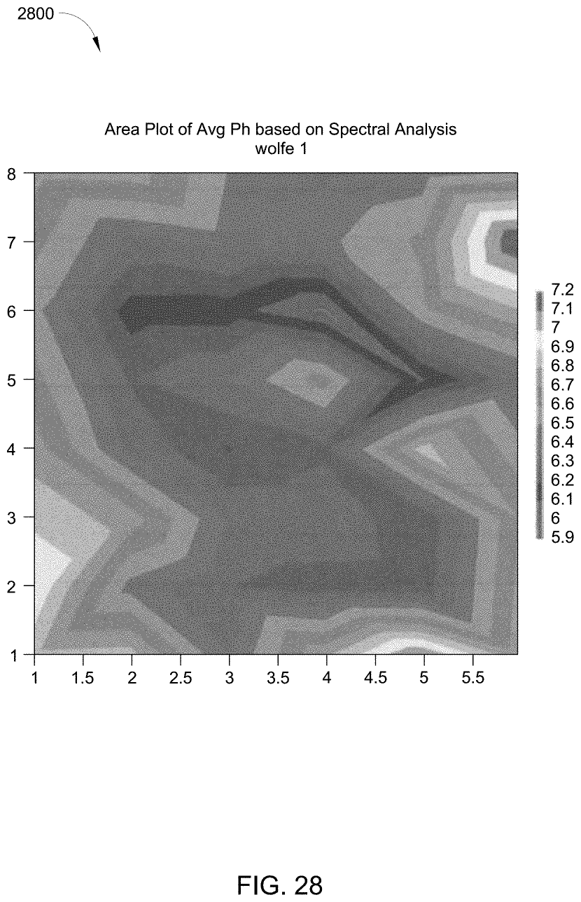

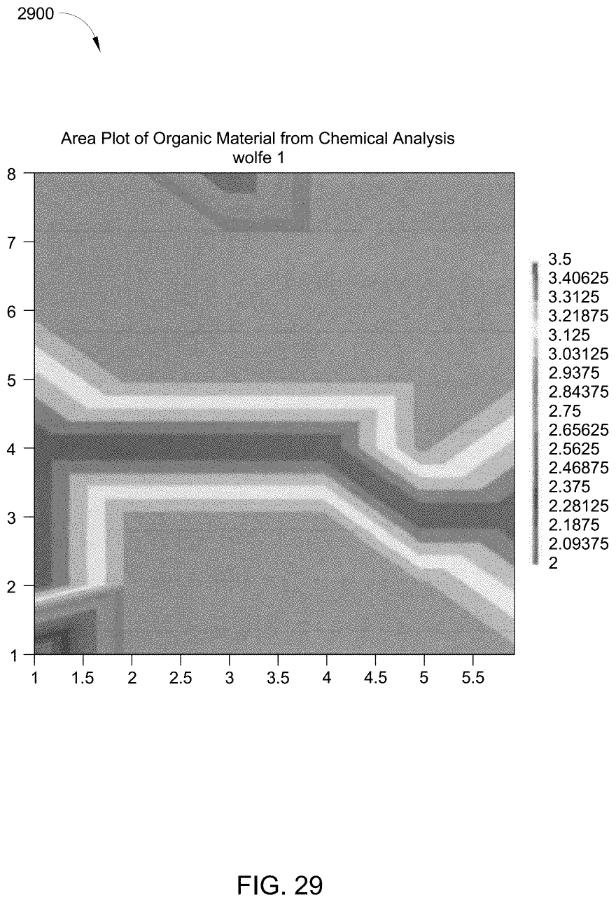

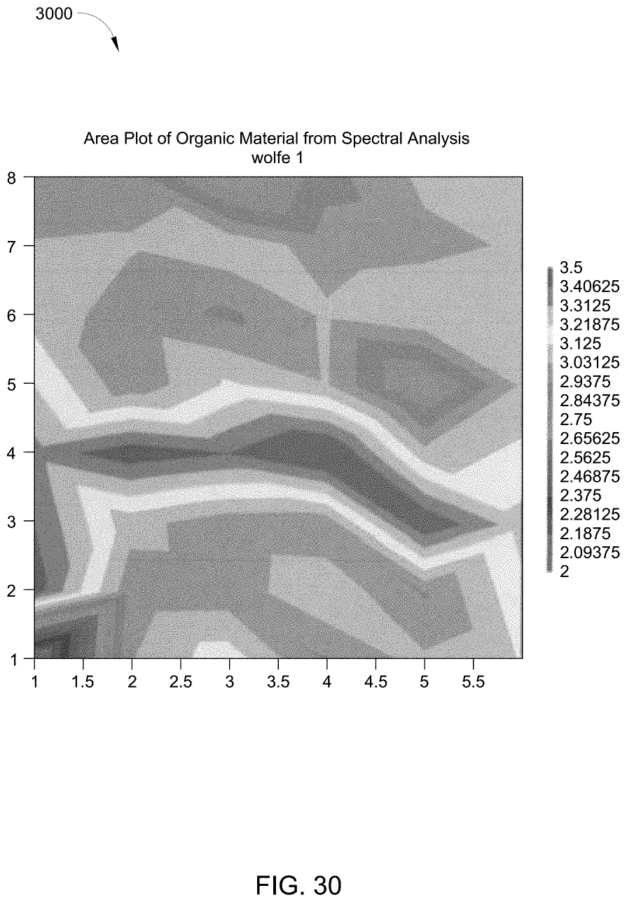

FIG. 21 illustrates an area plot (2100) of average nitrogen based on chemical analysis, in accordance with at least one embodiment. FIG. 22 illustrates an area plot (2200) of average nitrogen based on spectral data, in accordance with at least one embodiment. FIG. 23 illustrates an area plot (2300) of phosphorus based on chemical analysis, in accordance with at least one embodiment. FIG. 24 illustrates an area plot (2400) of phosphorus based on spectral analysis, in accordance with at least one embodiment. FIG. 25 illustrates an area plot (2500) of potassium based on chemical analysis, in accordance with at least one embodiment. FIG. 26 illustrates an area plot (2600) of potassium based on spectral analysis, in accordance with at least one embodiment. FIG. 27 illustrates an area plot (2700) of average phosphorus based on chemical analysis, in accordance with at least one embodiment. FIG. 28 illustrates an area plot (2800) of average Ph based on spectral analysis, in accordance with at least one embodiment. FIG. 29 illustrates an area plot (2900) of organic material from chemical analysis, in accordance with at least one embodiment. FIG. 30 illustrates an area plot (3000) of organic material from spectral analysis, in accordance with at least one embodiment.

This first test concluded that it is possible to measure N, P, and K in addition to other nutrients/elements by using spectrometer technology as long as one can understand the necessary spectral adjustments that need to be made in relation to the soil parameters such as the amount of moisture the soil type, the lighting and season at the time of test and others

The embodiments of the present invention have solved inconsistencies of spectral imaging for soil mapping capabilities by building adaptive hardware and sensors in combination with modeling analysis and information processing as compared to a growing database of acquired and chemically confirmed soil samples. The embodied improved processes, methods, and machinery for analyzing soil will create more rapid and accurate soil maps to more efficiently enable the user to determine soil content.

Soils are made up of four basic components: Sand, Silt, Clay, and Organic Matter. Basic information about their makeup in the soil can be found in the Soil Survey.

Organic Matter (OM) is made up of dead and decaying plants, animals, and microorganisms. OM is a repository of nutrients that are released into the soil as it decomposes. OM also has a large water holding capacity, which helps retain moisture in soils during times of drought. Primarily organic matter is found at the top and in the uppermost layers of the soil profile, where most root growth occurs.

Soil Texture indicates the relative content of particles of various sizes, such as sand, silt, and clay in the soil. Texture influences the ease with which soil can be worked, the amount of water and air it holds, and the rate at which water can enter and move through the soil.

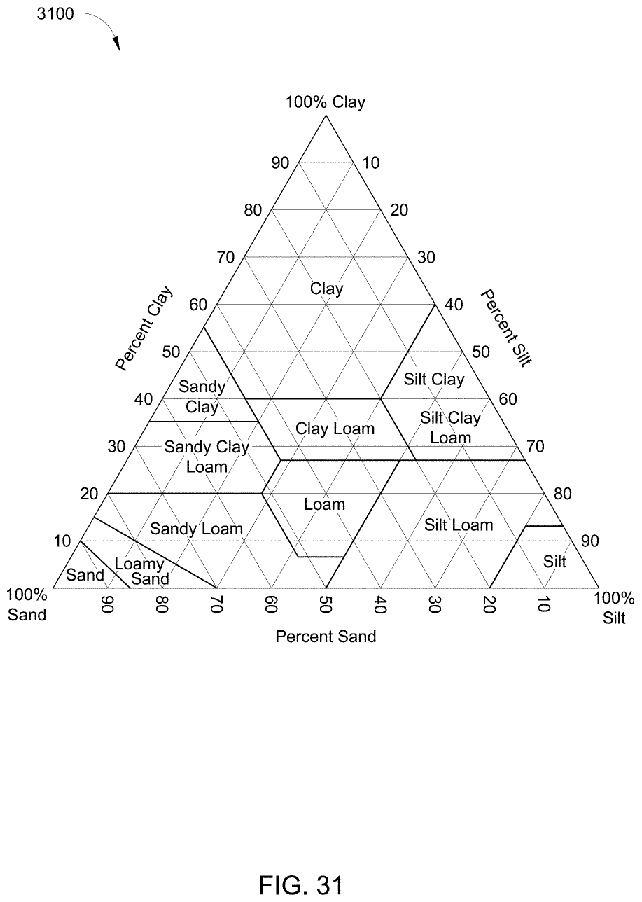

FIG. 31 illustrates a perspective view (3100) of a soil texture triangle, in accordance with at least one embodiment. Sand is the largest soil particle at 0.05 to 2 mm. Anything larger than that is considered to be gravel and stones. Sand, with its large diameter and low surface area to volume ratio, allows water to drain right through and cannot hold onto many nutrients. Silt is the middle soil particle at 0.002 to 0.05 mm. Silt is commonly found in waterways and floodplains. With some water holding capacity and some nutrient holding capacity, silt is part of a good soil mix with moderate drainage and nutrients. Clay is different from sand and silt in that it is made up of silicon, aluminum, and oxygen. It is the smallest soil particle at 0.002 mm or less and has a very high water-holding capacity and a high surface area to volume ratio enabling it to be a very good nutrient holder. Soils that hold water often include a lot of clay, and many plants are specially adapted to live in high clay soils. The ideal soil is considered to be a loam, which is a mix of sand, silt, and clay. Loams take advantage of the balance of water holding and nutrient availability between the three. Loamy soils with high organic matter are very well suited for crop production.

No language in the specification should be construed as indicating any non-claimed element as essential to the practice of the invention.

It will be apparent to those skilled in the art that various modifications and variations can be made to the present invention without departing from the spirit and scope of the invention. There is no intention to limit the invention to the specific form or forms enclosed. On the contrary, the intention is to cover all modifications, alternative constructions, and equivalents falling within the spirit and scope of the invention, as defined in the appended claims. Thus, it is intended that the present invention cover the modifications and variations of this invention, provided they are within the scope of the appended claims and their equivalents.

1. An apparatus to produce a plurality of soil maps, comprising:
a probe cylinder (1) configured to extend the probe (15) downward into the soil to an appropriate depth, and the probe cylinder (1) collects the sample core then retracts to the original starting position;
a first stepper motor (4) to move a collector assembly (3) to a position that centers the collector assembly (3) directly under the probe (15);
an extractor cylinder (2) to extend pushing the sample core into the collector assembly (3);
a mixing motor (6) is activated and then the second stepper motor (12) raises the collector assembly (3) into the mixer which pulverizes the sample core, wherein, once the collector assembly (3) reaches a top position, the second stepper motor (12) reverses lowering the collector assembly (3) to its original beginning position under the mixing motor;
wherein the first stepper motor (4) then moves the collector assembly (3) to a position centered directly under the contact probe (7), wherein the second stepper motor (12) then raises the collector assembly (3) to a position so that the contact probe (7) is inside the collector assembly (3) just above the mixed sample,
wherein the contact probe (7) scans the soil sample, and then the second stepper motor (12) reverses lowering the collector assembly (3) to its beginning position directly under the contact probe (7),
wherein the first stepper motor (4) then moves the collector assembly (3) to a position directly center under the brush motor (8) and over a bag holder conveyor,
wherein the third stepper motor (14) activates and opens a slide on the bottom of the collector assembly (3) allowing the soil sample to be deposited in a bag that is situated on the bag holder conveyor directly under the collector assembly (3),
wherein the brush motor (6) turns on at the same time the second stepper motor (12) activates raising the collector assembly (3) upward into a brush assembly, wherein, once the collector assembly (3) reaches its most upward position it reverses and lowers to its original starting position under the brush motor (8), wherein the brush motor (8) then stops and the third stepper motor (14) activates shutting the bottom slide on the collector assembly (3); and
one or more hyperspectral sensors configured to detect soil samples.

2. The apparatus as claimed in claim 1, wherein the hyperspectral sensors are placed in controlled halogen lighting conditions.

3. The apparatus as claimed in claim 1, wherein the soil samples are passed under the hyperspectral sensors.

4. The apparatus as claimed in claim 1, wherein the hyperspectral sensors have a range of 400-2400 nm.

5. The apparatus as claimed in claim 1, wherein the controlled halogen lighting conditions are 700 Watts and approximating daylight levels per square meter.

6. The apparatus as claimed in claim 1, wherein the extractor cylinder (2) begins to retract the first stepper motor (4) activates moving the collector assembly (3) to a position directly centered under the mixing motor (6).

7. The apparatus as claimed in claim 1, wherein the brush assembly consists of fiber brush the same size as the collector assembly (3) which cleans any soil residue off of the collector assembly (3).

8. The apparatus as claimed in claim 1, wherein the bag holder conveyor then advances to the next bag holder ready for a new sample and repeats the process.

9. The apparatus as claimed in claim 1 further comprises an image collector; a database, a processor, and an output device.

10. The apparatus as claimed in claim 9, wherein the database is associated with confirmed spectral images; the processor is configured to process the spectral images for adjusting the image based on field parameters, and the output device presents or displays soil map information.
